SEQUENCE LISTING (508 sequences)

SEQ ID NO: 1 - RNA, 22 nt, homo sapiens
ugagguaguagguuguauaguu

SEQ ID NO: 2 - RNA, 22 nt, homo sapiens
ugagguaguagguugugugguu

SEQ ID NO: 3 - RNA, 22 nt, homo sapiens
ugagguaguagguuguaugguu

SEQ ID NO: 4 - RNA, 22 nt, homo sapiens
agagguaguagguugcauaguu

SEQ ID NO: 5 - RNA, 22 nt, homo sapiens
ugagguaggagguuguauaguu

SEQ ID NO: 6 - RNA, 22 nt, homo sapiens
ugagguaguagauuguauaguu

SEQ ID NO: 7 - RNA, 22 nt, homo sapiens
ugagguaguaguuuguacaguu

SEQ ID NO: 8 - RNA, 22 nt, homo sapiens
ugagguaguaguuugugcuguu

SEQ ID NO: 9 - RNA, 22 nt, homo sapiens
uggaauguaaagaaguauguau

SEQ ID NO: 10 - RNA, 22 nt, homo sapiens
aacccguagauccgaacuugug

SEQ ID NO: 11 - RNA, 22 nt, homo sapiens
uacaguacugugauaacugaag

SEQ ID NO: 12 - RNA, 23 nt, homo sapiens
agcagcauuguacagggcuauga

SEQ ID NO: 13 - RNA, 23 nt, homo sapiens
ucaaaugcucagacuccuguggu

SEQ ID NO: 14 - RNA, 23 nt, homo sapiens
aaaagugcuuacagugcagguag

SEQ ID NO: 15 - RNA, 21 nt, homo sapiens
uaaagugcugacagugcagau

SEQ ID NO: 16 - RNA, 21 nt, homo sapiens
agcagcauuguacagggcuau

SEQ ID NO: 17 - RNA, 22 nt, homo sapiens
uacccuguagauccgaauuugu

SEQ ID NO: 18 - RNA, 22 nt, homo sapiens
uacccuguagaaccgaauuugu

SEQ ID NO: 19 - RNA, 22 nt, homo sapiens
aagcauucuuucauugguuggu

SEQ ID NO: 20 - RNA, 22 nt, homo sapiens
uuuccggcucgcgugggugugu

SEQ ID NO: 21 - RNA, 22 nt, homo sapiens
agaggauacccuuuguauguuc

SEQ ID NO: 22 - RNA, 23 nt, homo sapiens
gggaugguagaccggugacgugc

SEQ ID NO: 23 - RNA, 21 nt, homo sapiens
uaggacacauggucuacuucu

SEQ ID NO: 24 - RNA, 23 nt, homo sapiens
uggagugugacaaugguguuugu

SEQ ID NO: 25 - RNA, 21 nt, homo sapiens
uaaggcacgcggugaaugcca

SEQ ID NO: 26 - RNA, 21 nt, homo sapiens
aagugaucuaaaggccuacau

SEQ ID NO: 27 - RNA, 22 nt, homo sapiens
acccgucccguucguccccgga

SEQ ID NO: 28 - RNA, 22 nt, homo sapiens
acgcccuucccccccuucuuca

SEQ ID NO: 29 - RNA, 21 nt, homo sapiens
acggugcuggauguggccuuu

SEQ ID NO: 30 - RNA, 21 nt, homo sapiens
acucuagcugccaaaggcgcu

SEQ ID NO: 31 - RNA, 21 nt, homo sapiens
agaaggaaauugaauucauuu

SEQ ID NO: 32 - RNA, 23 nt, homo sapiens
aggaugagcaaagaaaguagauu

SEQ ID NO: 33 - RNA, 22 nt, homo sapiens
cggaugagcaaagaaagugguu

SEQ ID NO: 34 - RNA, 22 nt, homo sapiens
cuaaagagaagucaaugcauga

SEQ ID NO: 35 - RNA, 21 nt, homo sapiens
aguuaggauuaggucguggaa

SEQ ID NO: 36 - RNA, 22 nt, homo sapiens
ucccugagacccuuuaaccugu

SEQ ID NO: 37 - RNA, 22 nt, homo sapiens
ucccugagacccuaacuuguga

SEQ ID NO: 38 - RNA, 22 nt, homo sapiens
ucguaccgugaguaauaaugcg

SEQ ID NO: 39 - RNA, 22 nt, homo sapiens
augguacccuggcauacugagu

SEQ ID NO: 40 - RNA, 22 nt, homo sapiens
caagucuuauuugagcaccugu

SEQ ID NO: 41 - RNA, 23 nt, homo sapiens
ccucagggcuguagaacagggcu

SEQ ID NO: 42 - RNA, 22 nt, homo sapiens
cuggacugagccgugcuacugg

SEQ ID NO: 43 - RNA, 22 nt, homo sapiens
ucggauccgucugagcuuggcu

SEQ ID NO: 44 - RNA, 23 nt, homo sapiens
cuggagauauggaagagcugugu

SEQ ID NO: 45 - RNA, 22 nt, homo sapiens
cuuggcaccuagcaagcacuca

SEQ ID NO: 46 - RNA, 22 nt, homo sapiens
uacguagauauauauguauuuu

SEQ ID NO: 47 - RNA, 22 nt, homo sapiens
uaguacugugcauaucaucuau

SEQ ID NO: 48 - RNA, 21 nt, homo sapiens
ucacagugaaccggucucuuu

SEQ ID NO: 49 - RNA, 22 nt, homo sapiens
ucuacaaaggaaagcgcuuucu

SEQ ID NO: 50 - RNA, 22 nt, homo sapiens
gaaagcccauguuuguauugga

SEQ ID NO: 51 - RNA, 21 nt, homo sapiens
ugcaggaccaagaugagcccu

SEQ ID NO: 52 - RNA, 21 nt, homo sapiens
ugcuggaucagugguucgagu

SEQ ID NO: 53 - RNA, 22 nt, homo sapiens
uggaguccaggaaucugcauuu

SEQ ID NO: 54 - RNA, 22 nt, homo sapiens
aagcccuuaccccaaaaaguau

SEQ ID NO: 55 - RNA, 22 nt, homo sapiens
aagcccuuaccccaaaaagcau

SEQ ID NO: 56 - RNA, 22 nt, homo sapiens
ucuggguggucuggagauuugu

SEQ ID NO: 57 - RNA, 22 nt, homo sapiens
ugugagguuggcauuguugucu

SEQ ID NO: 58 - RNA, 21 nt, homo sapiens
uuaggccgcagaucuggguga

SEQ ID NO: 59 - RNA, 22 nt, homo sapiens
uuagggcccuggcuccaucucc

SEQ ID NO: 60 - RNA, 20 nt, homo sapiens
uucauucggcuguccagaug

SEQ ID NO: 61 - RNA, 24 nt, homo sapiens
uugcagcugccugggagugacuuc

SEQ ID NO: 62 - RNA, 23 nt, homo sapiens
uuuuagagacggggucuugcucu

SEQ ID NO: 63 - RNA, 22 nt, homo sapiens
cgguuugaggcuacagugagau

SEQ ID NO: 64 - RNA, 22 nt, homo sapiens
uuuucaacucuaaugggagaga

SEQ ID NO: 65 - RNA, 22 nt, homo sapiens
ccaccuccccugcaaacgucca

SEQ ID NO: 66 - RNA, 21 nt, homo sapiens
ucgaccggaccucgaccggcu

SEQ ID NO: 67 - RNA, 22 nt, homo sapiens
cagugcaauguuaaaagggcau

SEQ ID NO: 68 - RNA, 22 nt, homo sapiens
cagugcaaugaugaaagggcau

SEQ ID NO: 69 - RNA, 22 nt, homo sapiens
uaacagucuacagccauggucg

SEQ ID NO: 70 - RNA, 22 nt, homo sapiens
ucaaaacugaggggcauuuucu

SEQ ID NO: 71 - RNA, 23 nt, homo sapiens
uuugguccccuucaaccagcugu

SEQ ID NO: 72 - RNA, 21 nt, homo sapiens
uuugguccccuucaaccagcu

SEQ ID NO: 73 - RNA, 22 nt, homo sapiens
ugugacugguugaccagagggg

SEQ ID NO: 74 - RNA, 23 nt, homo sapiens
uauggcuuuuuauuccuauguga

SEQ ID NO: 75 - RNA, 23 nt, homo sapiens
uauggcuuuucauuccuauguga

SEQ ID NO: 76 - RNA, 23 nt, homo sapiens
acuccauuuguuuugaugaugga

SEQ ID NO: 77 - RNA, 23 nt, homo sapiens
uuauugcuuaagaauacgcguag

SEQ ID NO: 78 - RNA, 23 nt, homo sapiens
agcugguguugugaaucaggccg

SEQ ID NO: 79 - RNA, 23 nt, homo sapiens
ucuacagugcacgugucuccagu

SEQ ID NO: 80 - RNA, 22 nt, homo sapiens
accacaggguagaaccacggac

SEQ ID NO: 81 - RNA, 23 nt, homo sapiens
uaacacugucugguaaagauggc

SEQ ID NO: 82 - RNA, 23 nt, homo sapiens
uguaguguuuccuacuuuaugga

SEQ ID NO: 83 - RNA, 21 nt, homo sapiens
ugagaugaagcacuguagcuc

SEQ ID NO: 84 - RNA, 20 nt, homo sapiens
uacaguauagaugauguacu

SEQ ID NO: 85 - RNA, 23 nt, homo sapiens
guccaguuuucccaggaaucccu

SEQ ID NO: 86 - RNA, 22 nt, homo sapiens
cuccguuugccuguuucgcuga

SEQ ID NO: 87 - RNA, 22 nt, homo sapiens
ugagaacugaauuccauggguu

SEQ ID NO: 88 - RNA, 22 nt, homo sapiens
ugagaacugaauuccauaggcu

SEQ ID NO: 89 - RNA, 21 nt, homo sapiens
gugugcggaaaugcuucugcu

SEQ ID NO: 90 - RNA, 22 nt, homo sapiens
ucagugcacuacagaacuuugu

SEQ ID NO: 91 - RNA, 22 nt, homo sapiens
ucagugcaucacagaacuuugu

SEQ ID NO: 92 - RNA, 23 nt, homo sapiens
ucuggcuccgugucuucacuccc

SEQ ID NO: 93 - RNA, 22 nt, homo sapiens
ucucccaacccuuguaccagug

SEQ ID NO: 94 - RNA, 21 nt, homo sapiens
ucgaggagcucacagucuagu

SEQ ID NO: 95 - RNA, 21 nt, homo sapiens
ucagugcaugacagaacuugg

SEQ ID NO: 96 - RNA, 22 nt, homo sapiens
uugcauagucacaaaagugauc

SEQ ID NO: 97 - RNA, 22 nt, homo sapiens
aaaaccgucuaguuacaguugu

SEQ ID NO: 98 - RNA, 22 nt, homo sapiens
aaucauacacgguugaccuauu

SEQ ID NO: 99 - RNA, 23 nt, homo sapiens
uuaaugcuaaucgugauaggggu

SEQ ID NO: 100 - RNA, 21 nt, homo sapiens
uagcagcacauaaugguuugu

SEQ ID NO: 101 - RNA, 22 nt, homo sapiens
uagcagcacaucaugguuuaca

SEQ ID NO: 102 - RNA, 22 nt, homo sapiens
uagcagcacguaaauauuggcg

SEQ ID NO: 103 - RNA, 23 nt, homo sapiens
caaagugcuuacagugcagguag

SEQ ID NO: 104 - RNA, 23 nt, homo sapiens
aacauucaacgcugucggugagu

SEQ ID NO: 105 - RNA, 23 nt, homo sapiens
aacauucauugcugucggugggu

SEQ ID NO: 106 - RNA, 24 nt, homo sapiens
aacauucaaccugucggugaguuu

SEQ ID NO: 107 - RNA, 23 nt, homo sapiens
aacauucauuguugucggugggu

SEQ ID NO: 108 - RNA, 24 nt, homo sapiens
uuuggcaaugguagaacucacacu

SEQ ID NO: 109 - RNA, 22 nt, homo sapiens
uauggcacugguagaauucacu

SEQ ID NO: 110 - RNA, 22 nt, homo sapiens
uggacggagaacugauaagggu

SEQ ID NO: 111 - RNA, 22 nt, homo sapiens
uggagagaaaggcaguuccuga

SEQ ID NO: 112 - RNA, 22 nt, homo sapiens
caaagaauucuccuuuugggcu

SEQ ID NO: 113 - RNA, 22 nt, homo sapiens
ucgugucuuguguugcagccgg

SEQ ID NO: 114 - RNA, 22 nt, homo sapiens
caucccuugcaugguggagggu

SEQ ID NO: 115 - RNA, 23 nt, homo sapiens
uaaggugcaucuagugcagauag

SEQ ID NO: 116 - RNA, 21 nt, homo sapiens
uaaggugcaucuagugcaguu

SEQ ID NO: 117 - RNA, 21 nt, homo sapiens
cggcggggacggcgauugguc

SEQ ID NO: 118 - RNA, 23 nt, homo sapiens
ugauauguuugauauauuagguu

SEQ ID NO: 119 - RNA, 22 nt, homo sapiens
ugauauguuugauauuggguug

SEQ ID NO: 120 - RNA, 22 nt, homo sapiens
caacggaaucccaaaagcagcu

SEQ ID NO: 121 - RNA, 21 nt, homo sapiens
ccaguccugugccugccgccu

SEQ ID NO: 122 - RNA, 23 nt, homo sapiens
ugaguaccgccaugucuguuggg

SEQ ID NO: 123 - RNA, 21 nt, homo sapiens
ugcucauugcaugggcugugu

SEQ ID NO: 124 - RNA, 23 nt, homo sapiens
cccugugcccggcccacuucugc

SEQ ID NO: 125 - RNA, 21 nt, homo sapiens
cugaccuaugaauugacagcc

SEQ ID NO: 126 - RNA, 22 nt, homo sapiens
aacuggccuacaaagucccagu

SEQ ID NO: 127 - RNA, 22 nt, homo sapiens
aacuggcccucaaagucccgcu

SEQ ID NO: 128 - RNA, 22 nt, homo sapiens
uguaacagcaacuccaugugga

SEQ ID NO: 129 - RNA, 22 nt, homo sapiens
uagcagcacagaaauauuggca

SEQ ID NO: 130 - RNA, 22 nt, homo sapiens
uagguaguuucauguuguuggg

SEQ ID NO: 131 - RNA, 22 nt, homo sapiens
uagguaguuuccuguuguuggg

SEQ ID NO: 132 - RNA, 22 nt, homo sapiens
uucaccaccuucuccacccagc

SEQ ID NO: 133 - RNA, 21 nt, homo sapiens
acaguagucugcacauugguu

SEQ ID NO: 134 - RNA, 21 nt, homo sapiens
acaguagucugcacauugguu

SEQ ID NO: 135 - RNA, 23 nt, homo sapiens
ugugcaaaucuaugcaaaacuga

SEQ ID NO: 136 - RNA, 23 nt, homo sapiens
ugugcaaauccaugcaaaacuga

SEQ ID NO: 137 - RNA, 23 nt, homo sapiens
uaacacugucugguaacgauguu

SEQ ID NO: 138 - RNA, 22 nt, homo sapiens
uaauacugccugguaaugauga

SEQ ID NO: 139 - RNA, 23 nt, homo sapiens
uaauacugccggguaaugaugga

SEQ ID NO: 140 - RNA, 20 nt, homo sapiens
agagguauagggcaugggaa

SEQ ID NO: 141 - RNA, 22 nt, homo sapiens
gugaaauguuuaggaccacuag

SEQ ID NO: 142 - RNA, 22 nt, homo sapiens
uucccuuugucauccuaugccu

SEQ ID NO: 143 - RNA, 23 nt, homo sapiens
uccuucauuccaccggagucugu

SEQ ID NO: 144 - RNA, 22 nt, homo sapiens
uggaauguaaggaagugugugg

SEQ ID NO: 145 - RNA, 22 nt, homo sapiens
auaagacgagcaaaaagcuugu

SEQ ID NO: 146 - RNA, 22 nt, homo sapiens
auaagacgaacaaaagguuugu

SEQ ID NO: 147 - RNA, 23 nt, homo sapiens
uaaagugcuuauagugcagguag

SEQ ID NO: 148 - RNA, 23 nt, homo sapiens
caaagugcucauagugcagguag

SEQ ID NO: 149 - RNA, 23 nt, homo sapiens
uagcuuaucagacugauguugac

SEQ ID NO: 150 - RNA, 22 nt, homo sapiens
cugugcgugugacagcggcuga

SEQ ID NO: 151 - RNA, 22 nt, homo sapiens
uucccuuugucauccuucgccu

SEQ ID NO: 152 - RNA, 23 nt, homo sapiens
uuggggaaacggccgcugaguga

SEQ ID NO: 153 - RNA, 22 nt, homo sapiens
uagucccuuccuugaagcgguc

SEQ ID NO: 154 - RNA, 22 nt, homo sapiens
agcuuccaugacuccugaugga

SEQ ID NO: 155 - RNA, 21 nt, homo sapiens
uccucccaugccaagaacucc

SEQ ID NO: 156 - RNA, 23 nt, homo sapiens
accuuggcucuagacugcuuacu

SEQ ID NO: 157 - RNA, 22 nt, homo sapiens
ugccugucuacacuugcugugc

SEQ ID NO: 158 - RNA, 22 nt, homo sapiens
augaccuaugaauugacagaca

SEQ ID NO: 159 - RNA, 22 nt, homo sapiens
uaaucucagcuggcaacuguga

SEQ ID NO: 160 - RNA, 22 nt, homo sapiens
aaaucucugcaggcaaauguga

SEQ ID NO: 161 - RNA, 23 nt, homo sapiens
uacugcaucaggaacugauugga

SEQ ID NO: 162 - RNA, 21 nt, homo sapiens
uugugcuugaucuaaccaugu

SEQ ID NO: 163 - RNA, 21 nt, homo sapiens
ugauuguccaaacgcaauucu

SEQ ID NO: 164 - RNA, 22 nt, homo sapiens
agaauuguggcuggacaucugu

SEQ ID NO: 165 - RNA, 22 nt, homo sapiens
aagcugccaguugaagaacugu

SEQ ID NO: 166 - RNA, 22 nt, homo sapiens
agcuacauugucugcuggguuu

SEQ ID NO: 167 - RNA, 23 nt, homo sapiens
agcuacaucuggcuacugggucu

SEQ ID NO: 168 - RNA, 22 nt, homo sapiens
ugucaguuugucaaauacccca

SEQ ID NO: 169 - RNA, 22 nt, homo sapiens
caagucacuagugguuccguuu

SEQ ID NO: 170 - RNA, 22 nt, homo sapiens
gcccucugucaccuugcagacg

SEQ ID NO: 171 - RNA, 23 nt, homo sapiens
agcgcgggcugagcgcugccagu

SEQ ID NO: 172 - RNA, 22 nt, homo sapiens
gagagcaguguguguugccugg

SEQ ID NO: 173 - RNA, 22 nt, homo sapiens
auccccagauacaauggacaau

SEQ ID NO: 174 - RNA, 22 nt, homo sapiens
aucacauugccagggauuucca

SEQ ID NO: 175 - RNA, 21 nt, homo sapiens
aucacauugccagggauuacc

SEQ ID NO: 176 - RNA, 22 nt, homo sapiens
uggcucaguucagcaggaacag

SEQ ID NO: 177 - RNA, 22 nt, homo sapiens
cauugcacuugucucggucuga

SEQ ID NO: 178 - RNA, 22 nt, homo sapiens
uucaaguaauccaggauaggcu

SEQ ID NO: 179 - RNA, 22 nt, homo sapiens
uucaaguaauucaggauagguu

SEQ ID NO: 180 - RNA, 21 nt, homo sapiens
uucacaguggcuaaguuccgc

SEQ ID NO: 181 - RNA, 21 nt, homo sapiens
uucacaguggcuaaguucugc

SEQ ID NO: 182 - RNA, 22 nt, homo sapiens
aaggagcucacagucuauugag

SEQ ID NO: 183 - RNA, 22 nt, homo sapiens
gaggguuggguggaggcucucc

SEQ ID NO: 184 - RNA, 22 nt, homo sapiens
ugguuuaccgucccacauacau

SEQ ID NO: 185 - RNA, 22 nt, homo sapiens
uagcaccaucugaaaucgguua

SEQ ID NO: 186 - RNA, 23 nt, homo sapiens
uagcaccauuugaaaucaguguu

SEQ ID NO: 187 - RNA, 21 nt, homo sapiens
uagcaccauuugaaaucgguu

SEQ ID NO: 188 - RNA, 23 nt, homo sapiens
cagugcaauaguauugucaaagc

SEQ ID NO: 189 - RNA, 23 nt, homo sapiens
cagugcaaugauauugucaaagc

SEQ ID NO: 190 - RNA, 22 nt, homo sapiens
uaaacguggauguacuugcuuu

SEQ ID NO: 191 - RNA, 23 nt, homo sapiens
uaagugcuuccauguuuuaguag

SEQ ID NO: 192 - RNA, 22 nt, homo sapiens
aagugcuuccauguuucagugg

SEQ ID NO: 193 - RNA, 23 nt, homo sapiens
uaagugcuuccauguuugagugu

SEQ ID NO: 194 - RNA, 23 nt, homo sapiens
ucaacaaaaucacugaugcugga

SEQ ID NO: 195 - RNA, 22 nt, homo sapiens
guuccugcugaacugagccagu

SEQ ID NO: 196 - RNA, 24 nt, homo sapiens
uguaaacauccucgacuggaagcu

SEQ ID NO: 197 - RNA, 22 nt, homo sapiens
uguaaacauccuacacucagcu

SEQ ID NO: 198 - RNA, 24 nt, homo sapiens
uguaaacauccuacacucucagcu

SEQ ID NO: 199 - RNA, 24 nt, homo sapiens
uguaaacauccccgacuggaagcu

SEQ ID NO: 200 - RNA, 24 nt, homo sapiens
uguaaacauccuugacuggaagcu

SEQ ID NO: 201 - RNA, 23 nt, homo sapiens
ugcuaugccaacauauugccauc

SEQ ID NO: 202 - RNA, 20 nt, homo sapiens
auauggguuuacuaguuggu

SEQ ID NO: 203 - RNA, 22 nt, homo sapiens
auaggacucauauagugccagg

SEQ ID NO: 204 - RNA, 21 nt, homo sapiens
cacagcaaguguagacaggca

SEQ ID NO: 205 - RNA, 21 nt, homo sapiens
uucgcgggcgaaggcaaaguc

SEQ ID NO: 206 - RNA, 22 nt, homo sapiens
ugagggacagaugccagaagca

SEQ ID NO: 207 - RNA, 23 nt, homo sapiens
aucagggcuuguggaaugggaag

SEQ ID NO: 208 - RNA, 22 nt, homo sapiens
aaacuaaucucuacacugcugc

SEQ ID NO: 209 - RNA, 21 nt, homo sapiens
gcugcaccggagacuggguaa

SEQ ID NO: 210 - RNA, 22 nt, homo sapiens
cugacugaauagguagggucau

SEQ ID NO: 211 - RNA, 21 nt, homo sapiens
acagugagguagagggagugc

SEQ ID NO: 212 - RNA, 23 nt, homo sapiens
uaggagcucaacagaugccuguu

SEQ ID NO: 213 - RNA, 21 nt, homo sapiens
agcuuuugggaauucagguag

SEQ ID NO: 214 - RNA, 22 nt, homo sapiens
auaacauuguaaagcgcuucuu

SEQ ID NO: 215 - RNA, 21 nt, homo sapiens
auauaccuguucggucucuuu

SEQ ID NO: 216 - RNA, 22 nt, homo sapiens
aacuccaaacacucaaaacuca

SEQ ID NO: 217 - RNA, 23 nt, homo sapiens
caugcuaggauagaaagaauggg

SEQ ID NO: 218 - RNA, 23 nt, homo sapiens
uuuguauggauauguguguguau

SEQ ID NO: 219 - RNA, 22 nt, homo sapiens
caaccucgacgaucuccucagc

SEQ ID NO: 220 - RNA, 21 nt, homo sapiens
gguggggcaaugggaucaggu

SEQ ID NO: 221 - RNA, 22 nt, homo sapiens
auugccucuguucuaacacaag

SEQ ID NO: 222 - RNA, 22 nt, homo sapiens
ccucccacugcagagccugggg

SEQ ID NO: 223 - RNA, 22 nt, homo sapiens
uucagccaggcuagugcagucu

SEQ ID NO: 224 - RNA, 22 nt, homo sapiens
aagggcuuccucucugcaggac

SEQ ID NO: 225 - RNA, 22 nt, homo sapiens
cugggguucugagacagacagu

SEQ ID NO: 226 - RNA, 22 nt, homo sapiens
uauauagauuccauaaaucuau

SEQ ID NO: 227 - RNA, 22 nt, homo sapiens
ugcccugccuguuuucuccuuu

SEQ ID NO: 228 - RNA, 22 nt, homo sapiens
uagugaguuagagaugcagagc

SEQ ID NO: 229 - RNA, 22 nt, homo sapiens
cggggagagaacgcagugacgu

SEQ ID NO: 230 - RNA, 21 nt, homo sapiens
acuggccugggacuaccgggg

SEQ ID NO: 231 - RNA, 21 nt, homo sapiens
ugcacggcacuggggacacgu

SEQ ID NO: 232 - RNA, 22 nt, homo sapiens
gccucucucggagucgcucgga

SEQ ID NO: 233 - RNA, 20 nt, homo sapiens
uuggccauggggcugcgcgg

SEQ ID NO: 234 - RNA, 22 nt, homo sapiens
cccuugggucugaugggguagc

SEQ ID NO: 235 - RNA, 22 nt, homo sapiens
uccugcguaggaucugaggagu

SEQ ID NO: 236 - RNA, 22 nt, homo sapiens
ggccagccaccaggagggcugc

SEQ ID NO: 237 - RNA, 22 nt, homo sapiens
guggaguccuggggaauggaga

SEQ ID NO: 238 - RNA, 21 nt, homo sapiens
uauugcacauuacuaaguugc

SEQ ID NO: 239 - RNA, 22 nt, homo sapiens
aaaagcuggguugagagggcga

SEQ ID NO: 240 - RNA, 23 nt, homo sapiens
caccuugcgcuacucaggucugc

SEQ ID NO: 241 - RNA, 22 nt, homo sapiens
gcacauuacacggucgaccucu

SEQ ID NO: 242 - RNA, 21 nt, homo sapiens
cccaauacacggucgaccucu

SEQ ID NO: 243 - RNA, 23 nt, homo sapiens
cgcauccccuagggcauuggugu

SEQ ID NO: 244 - RNA, 22 nt, homo sapiens
uuuauugaggaccuccuaucaa

SEQ ID NO: 245 - RNA, 20 nt, homo sapiens
ccucugggcccuuccuccag

SEQ ID NO: 246 - RNA, 22 nt, homo sapiens
cuggcccucucugcccuuccgu

SEQ ID NO: 247 - RNA, 22 nt, homo sapiens
aacacaccugguuaaccucuuu

SEQ ID NO: 248 - RNA, 23 nt, homo sapiens
gcaaagcacacggccugcagaga

SEQ ID NO: 249 - RNA, 20 nt, homo sapiens
gccccugggccuauccuaga

SEQ ID NO: 250 - RNA, 22 nt, homo sapiens
ucaagagcaauaacgaaaaaug

SEQ ID NO: 251 - RNA, 21 nt, homo sapiens
uccuauaugaugccuuucuuc

SEQ ID NO: 252 - RNA, 21 nt, homo sapiens
uccagcaucagugauuuuguu

SEQ ID NO: 253 - RNA, 23 nt, homo sapiens
ucccuguccuccaggagcucacg

SEQ ID NO: 254 - RNA, 20 nt, homo sapiens
gugcauuguaguugcauugc

SEQ ID NO: 255 - RNA, 20 nt, homo sapiens
gugcauugcuguugcauugc

SEQ ID NO: 256 - RNA, 22 nt, homo sapiens
uuauaaagcaaugagacugauu

SEQ ID NO: 257 - RNA, 23 nt, homo sapiens
ucucacacagaaaucgcacccgu

SEQ ID NO: 258 - RNA, 21 nt, homo sapiens
gcugacuccuaguccagggcu

SEQ ID NO: 259 - RNA, 23 nt, homo sapiens
ugucugcccgcaugccugccucu

SEQ ID NO: 260 - RNA, 22 nt, homo sapiens
uggcagugucuuagcugguugu

SEQ ID NO: 261 - RNA, 23 nt, homo sapiens
aggcagugucauuagcugauugu

SEQ ID NO: 262 - RNA, 23 nt, homo sapiens
aggcaguguaguuagcugauugc

SEQ ID NO: 263 - RNA, 21 nt, homo sapiens
ccuccguguuaccuguccucu

SEQ ID NO: 264 - RNA, 22 nt, homo sapiens
uuaucagaaucuccagggguac

SEQ ID NO: 265 - RNA, 20 nt, homo sapiens
uugugaagaaagaaauucuu

SEQ ID NO: 266 - RNA, 22 nt, homo sapiens
aggaggcaucuugagaaaugga

SEQ ID NO: 267 - RNA, 22 nt, homo sapiens
uguuguacuuuuuuuuuuguuc

SEQ ID NO: 268 - RNA, 22 nt, homo sapiens
uagccuucagaucuugguguuu

SEQ ID NO: 269 - RNA, 22 nt, homo sapiens
aaagacauaguugcaagauggg

SEQ ID NO: 270 - RNA, 21 nt, homo sapiens
ucagcaggcaggcuggugcag

SEQ ID NO: 271 - RNA, 24 nt, homo sapiens
aauccuuggaaccuaggugugagu

SEQ ID NO: 272 - RNA, 22 nt, homo sapiens
caggcacgggagcucaggugag

SEQ ID NO: 273 - RNA, 22 nt, homo sapiens
aauugcacgguauccaucugua

SEQ ID NO: 274 - RNA, 22 nt, homo sapiens
uaaugccccuaaaaauccuuau

SEQ ID NO: 275 - RNA, 22 nt, homo sapiens
uugugucccauuauuggugauu

SEQ ID NO: 276 - RNA, 22 nt, homo sapiens
ugaguguugucuacgagggcau

SEQ ID NO: 277 - RNA, 22 nt, homo sapiens
aacucugucuucacucaugagu

SEQ ID NO: 278 - RNA, 22 nt, homo sapiens
accuuccucuccaugggucuuu

SEQ ID NO: 279 - RNA, 22 nt, homo sapiens
aauugcacuuuagcaaugguga

SEQ ID NO: 280 - RNA, 22 nt, homo sapiens
cucgugggcucuggccacggcc

SEQ ID NO: 281 - RNA, 19 nt, homo sapiens
ugaggauauggcagggaag

SEQ ID NO: 282 - RNA, 21 nt, homo sapiens
acucacucacaggauugugca

SEQ ID NO: 283 - RNA, 22 nt, homo sapiens
uaguggaugaugcacucugugc

SEQ ID NO: 284 - RNA, 21 nt, homo sapiens
ugaugauacagguggagguag

SEQ ID NO: 285 - RNA, 22 nt, homo sapiens
uauggaaagacuuugccacucu

SEQ ID NO: 286 - RNA, 21 nt, homo sapiens
aauaauacaugguugaucuuu

SEQ ID NO: 287 - RNA, 22 nt, homo sapiens
uaguggaugauggagacucggu

SEQ ID NO: 288 - RNA, 22 nt, homo sapiens
gccugcugggguggaaccuggu

SEQ ID NO: 289 - RNA, 21 nt, homo sapiens
acucaaacugugggggcacuu

SEQ ID NO: 290 - RNA, 23 nt, homo sapiens
aaagugcugcgacauuugagcgu

SEQ ID NO: 291 - RNA, 23 nt, homo sapiens
gaagugcuucgauuuuggggugu

SEQ ID NO: 292 - RNA, 22 nt, homo sapiens
uuauaauacaaccugauaagug

SEQ ID NO: 293 - RNA, 22 nt, homo sapiens
auauaauacaaccugcuaagug

SEQ ID NO: 294 - RNA, 22 nt, homo sapiens
uuuguucguucggcucgcguga

SEQ ID NO: 295 - RNA, 21 nt, homo sapiens
aucauagaggaaaauccacgu

SEQ ID NO: 296 - RNA, 21 nt, homo sapiens
aucauagaggaaaauccaugu

SEQ ID NO: 297 - RNA, 21 nt, homo sapiens
aacauagaggaaauuccacgu

SEQ ID NO: 298 - RNA, 22 nt, homo sapiens
aucacacaaaggcaacuuuugu

SEQ ID NO: 299 - RNA, 22 nt, homo sapiens
acuggacuuggagucagaaggc

SEQ ID NO: 300 - RNA, 21 nt, homo sapiens
ugguagacuauggaacguagg

SEQ ID NO: 301 - RNA, 21 nt, homo sapiens
uauguaauaugguccacaucu

SEQ ID NO: 302 - RNA, 22 nt, homo sapiens
uauacaagggcaagcucucugu

SEQ ID NO: 303 - RNA, 22 nt, homo sapiens
gaaguuguucgugguggauucg

SEQ ID NO: 304 - RNA, 22 nt, homo sapiens
agaucagaaggugauuguggcu

SEQ ID NO: 305 - RNA, 22 nt, homo sapiens
auuccuagaaauuguucauaau

SEQ ID NO: 306 - RNA, 22 nt, homo sapiens
uguccucuagggccugcagucu

SEQ ID NO: 307 - RNA, 21 nt, homo sapiens
aaaaggcauaaaaccaagaca

SEQ ID NO: 308 - RNA, 21 nt, homo sapiens
uaacgcauaauauggacaugu

SEQ ID NO: 309 - RNA, 22 nt, homo sapiens
uacugaguccuuuguucucuac

SEQ ID NO: 310 - RNA, 22 nt, homo sapiens
ugugggacuucuggccuugacu

SEQ ID NO: 311 - RNA, 22 nt, homo sapiens
ggaggaaccuuggagcuucggc

SEQ ID NO: 312 - RNA, 22 nt, homo sapiens
ucagguguggaaacugaggcag

SEQ ID NO: 313 - RNA, 22 nt, homo sapiens
aauucccuuguagauaacccgg

SEQ ID NO: 314 - RNA, 22 nt, homo sapiens
uacgcgcagaccacaggauguc

SEQ ID NO: 315 - RNA, 21 nt, homo sapiens
cagcccggaucccagcccacu

SEQ ID NO: 316 - RNA, 21 nt, homo sapiens
agcaauacuguuaccugaaau

SEQ ID NO: 317 - RNA, 21 nt, homo sapiens
ugugcagcaggccaaccgaga

SEQ ID NO: 318 - RNA, 22 nt, homo sapiens
gaauguugcucggugaaccccu

SEQ ID NO: 319 - RNA, 21 nt, homo sapiens
aauauaacacagauggccugu

SEQ ID NO: 320 - RNA, 22 nt, homo sapiens
auaguagaccguauagcguacg

SEQ ID NO: 321 - RNA, 20 nt, homo sapiens
uucaccugguccacuagccg

SEQ ID NO: 322 - RNA, 23 nt, homo sapiens
aucaacagacauuaauugggcgc

SEQ ID NO: 323 - RNA, 23 nt, homo sapiens
agcucggucugaggccccucagu

SEQ ID NO: 324 - RNA, 21 nt, homo sapiens
cagcagcaauucauguuuuga

SEQ ID NO: 325 - RNA, 25 nt, homo sapiens
aaugacacgaucacucccguugagu

SEQ ID NO: 326 - RNA, 22 nt, homo sapiens
uaauacugucugguaaaaccgu

SEQ ID NO: 327 - RNA, 21 nt, homo sapiens
ugucuugcaggccgucaugca

SEQ ID NO: 328 - RNA, 23 nt, homo sapiens
ucuuggaguaggucauugggugg

SEQ ID NO: 329 - RNA, 23 nt, homo sapiens
uguuccucugucucccagacucu

SEQ ID NO: 330 - RNA, 22 nt, homo sapiens
aucaugaugggcuccucggugu

SEQ ID NO: 331 - RNA, 21 nt, homo sapiens
uugcauauguaggauguccca

SEQ ID NO: 332 - RNA, 22 nt, homo sapiens
uggcaguguauuguuagcuggu

SEQ ID NO: 333 - RNA, 23 nt, homo sapiens
aggcaguguauuguuagcuggcu

SEQ ID NO: 334 - RNA, 22 nt, homo sapiens
caguugcuaguugcacuccucu

SEQ ID NO: 335 - RNA, 22 nt, homo sapiens
uuuugcgauguguuccuaauau

SEQ ID NO: 336 - RNA, 22 nt, homo sapiens
uuuugcaauauguuccugaaua

SEQ ID NO: 337 - RNA, 19 nt, homo sapiens
aaaccguuaccauuacuga

SEQ ID NO: 338 - RNA, 22 nt, homo sapiens
aacuguuugcagaggaaacuga

SEQ ID NO: 339 - RNA, 23 nt, homo sapiens
uagugcaauauugcuuauagggu

SEQ ID NO: 340 - RNA, 22 nt, homo sapiens
uaugugccuuuggacuacaucg

SEQ ID NO: 341 - RNA, 22 nt, homo sapiens
uguguugcauguguguauaugu

SEQ ID NO: 342 - RNA, 22 nt, homo sapiens
cacuccucuccucccgucuucu

SEQ ID NO: 343 - RNA, 22 nt, homo sapiens
ccggggggggcggggccucgcg

SEQ ID NO: 344 - RNA, 22 nt, homo sapiens
gucauacacggcucuccucucu

SEQ ID NO: 345 - RNA, 22 nt, homo sapiens
uccuguacugagcugccccgag

SEQ ID NO: 346 - RNA, 22 nt, homo sapiens
aaucauacagggacauccaguu

SEQ ID NO: 347 - RNA, 22 nt, homo sapiens
aaucguacagggucauccacuu

SEQ ID NO: 348 - RNA, 21 nt, homo sapiens
uugaaaggcuauuucuugguc

SEQ ID NO: 349 - RNA, 22 nt, homo sapiens
gugacaucacauauacggcagc

SEQ ID NO: 350 - RNA, 20 nt, homo sapiens
ccauggaucuccaggugggu

SEQ ID NO: 351 - RNA, 23 nt, homo sapiens
aguggggaacccuuccaugagga

SEQ ID NO: 352 - RNA, 22 nt, homo sapiens
uuguacaugguaggcuuucauu

SEQ ID NO: 353 - RNA, 23 nt, homo sapiens
ugaaacauacacgggaaaccucu

SEQ ID NO: 354 - RNA, 22 nt, homo sapiens
aaacaaacauggugcacuucuu

SEQ ID NO: 355 - RNA, 22 nt, homo sapiens
gguuguccaugguguguucauu

SEQ ID NO: 356 - RNA, 21 nt, homo sapiens
cagcagcacacugugguuugu

SEQ ID NO: 357 - RNA, 23 nt, homo sapiens
uuucaagccagggggcguuuuuc

SEQ ID NO: 358 - RNA, 21 nt, homo sapiens
uuaagacuugcagugauguuu

SEQ ID NO: 359 - RNA, 23 nt, homo sapiens
augcaccugggcaaggauucuga

SEQ ID NO: 360 - RNA, 23 nt, homo sapiens
uaauccuugcuaccugggugaga

SEQ ID NO: 361 - RNA, 22 nt, homo sapiens
aaugcacccgggcaaggauucu

SEQ ID NO: 362 - RNA, 22 nt, homo sapiens
aaugcaccugggcaaggauuca

SEQ ID NO: 363 - RNA, 23 nt, homo sapiens
uagcagcgggaacaguucugcag

SEQ ID NO: 364 - RNA, 21 nt, homo sapiens
gacccuggucugcacucuauc

SEQ ID NO: 365 - RNA, 22 nt, homo sapiens
cgucaacacuugcugguuuccu

SEQ ID NO: 366 - RNA, 22 nt, homo sapiens
guaaggcacccuucugaguaga

SEQ ID NO: 367 - RNA, 23 nt, homo sapiens
ugauuguagccuuuuggaguaga

SEQ ID NO: 368 - RNA, 23 nt, homo sapiens
ugauugguacgucuguggguaga

SEQ ID NO: 369 - RNA, 23 nt, homo sapiens
ugauugaaaccucuaagagugga

SEQ ID NO: 370 - RNA, 21 nt, homo sapiens
gugucuuuugcucugcaguca

SEQ ID NO: 371 - RNA, 22 nt, homo sapiens
aagugcugucauagcugagguc

SEQ ID NO: 372 - RNA, 23 nt, homo sapiens
uaaauuucaccuuucugagaagg

SEQ ID NO: 373 - RNA, 22 nt, homo sapiens
uucacaaggaggugucauuuau

SEQ ID NO: 374 - RNA, 22 nt, homo sapiens
uucucaaggaggugucguuuau

SEQ ID NO: 375 - RNA, 21 nt, homo sapiens
auugacacuucugugaguaga

SEQ ID NO: 376 - RNA, 22 nt, homo sapiens
uucucaagagggaggcaaucau

SEQ ID NO: 377 - RNA, 22 nt, homo sapiens
gagugccuucuuuuggagcguu

SEQ ID NO: 378 - RNA, 22 nt, homo sapiens
uucucgaggaaagaagcacuuu

SEQ ID NO: 379 - RNA, 24 nt, homo sapiens
aucuggagguaagaagcacuuucu

SEQ ID NO: 380 - RNA, 22 nt, homo sapiens
aucuggagguaagaagcacuuu

SEQ ID NO: 381 - RNA, 22 nt, homo sapiens
aucgugcaucccuuuagagugu

SEQ ID NO: 382 - RNA, 22 nt, homo sapiens
aucgugcauccuuuuagagugu

SEQ ID NO: 383 - RNA, 22 nt, homo sapiens
gaaagcgcuucccuuugcugga

SEQ ID NO: 384 - RNA, 22 nt, homo sapiens
caaagcgcuccccuuuagaggu

SEQ ID NO: 385 - RNA, 23 nt, homo sapiens
caaagcgcuucucuuuagagugu

SEQ ID NO: 386 - RNA, 22 nt, homo sapiens
caaagcgcuucccuuuggagcg

SEQ ID NO: 387 - RNA, 22 nt, homo sapiens
aaagcgcuucccuucagagugu

SEQ ID NO: 388 - RNA, 22 nt, homo sapiens
gaaagcgcuucucuuuagagga

SEQ ID NO: 389 - RNA, 22 nt, homo sapiens
aaagugcauccuuuuagagugu

SEQ ID NO: 390 - RNA, 22 nt, homo sapiens
aaagugcauccuuuuagagguu

SEQ ID NO: 391 - RNA, 22 nt, homo sapiens
aaagugcaucuuuuuagaggau

SEQ ID NO: 392 - RNA, 23 nt, homo sapiens
caaagugccucccuuuagagugu

SEQ ID NO: 393 - RNA, 22 nt, homo sapiens
uucuccaaaagggagcacuuuc

SEQ ID NO: 394 - RNA, 21 nt, homo sapiens
cuccagagggaaguacuuucu

SEQ ID NO: 395 - RNA, 22 nt, homo sapiens
aaagugcuuccuuuuagagggu

SEQ ID NO: 396 - RNA, 22 nt, homo sapiens
aaagugcuuccuuuuagagggu

SEQ ID NO: 397 - RNA, 22 nt, homo sapiens
aaagugcuucucuuuggugggu

SEQ ID NO: 398 - RNA, 22 nt, homo sapiens
aaagugcuuccuuuuugagggu

SEQ ID NO: 399 - RNA, 22 nt, homo sapiens
caagugcuuccuuuuagagggu

SEQ ID NO: 400 - RNA, 24 nt, homo sapiens
acaaagugcuucccuuuagagugu

SEQ ID NO: 401 - RNA, 22 nt, homo sapiens
aaagugcuucccuuuagaguua

SEQ ID NO: 402 - RNA, 22 nt, homo sapiens
aacgcacuucccuuuagagugu

SEQ ID NO: 403 - RNA, 22 nt, homo sapiens
aaaaugguucccuuuagagugu

SEQ ID NO: 404 - RNA, 22 nt, homo sapiens
aacgcgcuucccuauagagggu

SEQ ID NO: 405 - RNA, 22 nt, homo sapiens
cuacaaagggaagcacuuucuc

SEQ ID NO: 406 - RNA, 22 nt, homo sapiens
cuccagagggaugcacuuucuc

SEQ ID NO: 407 - RNA, 22 nt, homo sapiens
gaaagcgcuuccuuuuagagga

SEQ ID NO: 408 - RNA, 22 nt, homo sapiens
gaacaugcauccuuucagaggg

SEQ ID NO: 409 - RNA, 23 nt, homo sapiens
cucuugagggaagcacuuucugu

SEQ ID NO: 410 - RNA, 21 nt, homo sapiens
cugcaaagggaagcccuuucu

SEQ ID NO: 411 - RNA, 22 nt, homo sapiens
caugccuugaguguaggaccgu

SEQ ID NO: 412 - RNA, 22 nt, homo sapiens
aucauacaaggacaauuucuuu

SEQ ID NO: 413 - RNA, 22 nt, homo sapiens
uggugggcacagaaucuggacu

SEQ ID NO: 414 - RNA, 22 nt, homo sapiens
ugugacagauugauaacugaaa

SEQ ID NO: 415 - RNA, 22 nt, homo sapiens
aaacauucgcggugcacuucuu

SEQ ID NO: 416 - RNA, 21 nt, homo sapiens
ucuuguuaaaaagcagauucu

SEQ ID NO: 417 - RNA, 22 nt, homo sapiens
ucaguaaauguuuauuagauga

SEQ ID NO: 418 - RNA, 22 nt, homo sapiens
agcucauccauaguugucacug

SEQ ID NO: 419 - RNA, 22 nt, homo sapiens
ugucuuacucccucaggcacau

SEQ ID NO: 420 - RNA, 20 nt, homo sapiens
gcgacccacucuugguuucc

SEQ ID NO: 421 - RNA, 21 nt, homo sapiens
gcgacccauacuugguuucag

SEQ ID NO: 422 - RNA, 21 nt, homo sapiens
aacaggugacugguuagacaa

SEQ ID NO: 423 - RNA, 21 nt, homo sapiens
gaugagcucauuguaauauga

SEQ ID NO: 424 - RNA, 22 nt, homo sapiens
uuuggugcauauuuacuuuagg

SEQ ID NO: 425 - RNA, 22 nt, homo sapiens
aucaaggaucuuaaacuuugcc

SEQ ID NO: 426 - RNA, 22 nt, homo sapiens
cgaaaacagcaauuaccuuugc

SEQ ID NO: 427 - RNA, 22 nt, homo sapiens
cacgcucaugcacacacccaca

SEQ ID NO: 428 - RNA, 22 nt, homo sapiens
auucuaauuucuccacgucuuu

SEQ ID NO: 429 - RNA, 22 nt, homo sapiens
guagauaaaauauugguaccug

SEQ ID NO: 430 - RNA, 23 nt, homo sapiens
uucauuugguauaaaccgcgauu

SEQ ID NO: 431 - RNA, 22 nt, homo sapiens
uugagaaugaugaaucauuagg

SEQ ID NO: 432 - RNA, 21 nt, homo sapiens
ucuuguguucucuagaucagu

SEQ ID NO: 433 - RNA, 23 nt, homo sapiens
uuacaguuguucaaccaguuacu

SEQ ID NO: 434 - RNA, 21 nt, homo sapiens
uuaugguuugccugggacuga

SEQ ID NO: 435 - RNA, 22 nt, homo sapiens
ugggcguaucuguaugcuaggg

SEQ ID NO: 436 - RNA, 21 nt, homo sapiens
uuggccacaauggguuagaac

SEQ ID NO: 437 - RNA, 21 nt, homo sapiens
ugagaaccacgucugcucuga

SEQ ID NO: 438 - RNA, 22 nt, homo sapiens
gagcuuauucauaaaagugcag

SEQ ID NO: 439 - RNA, 22 nt, homo sapiens
uugugucaauaugcgaugaugu

SEQ ID NO: 440 - RNA, 22 nt, homo sapiens
ugugucacucgaugaccacugu

SEQ ID NO: 441 - RNA, 22 nt, homo sapiens
uacgucaucguugucaucguca

SEQ ID NO: 442 - RNA, 22 nt, homo sapiens
uuugauaagcugacaugggaca

SEQ ID NO: 443 - RNA, 22 nt, homo sapiens
agaaggcacuaugagauuuaga

SEQ ID NO: 444 - RNA, 21 nt, homo sapiens
ugagcuaaaugugugcuggga

SEQ ID NO: 445 - RNA, 21 nt, homo sapiens
uccgagccugggucucccucu

SEQ ID NO: 446 - RNA, 22 nt, homo sapiens
acucaaaacccuucagugacuu

SEQ ID NO: 447 - RNA, 23 nt, homo sapiens
aaacucuacuuguccuucugagu

SEQ ID NO: 448 - RNA, 21 nt, homo sapiens
uaguaccaguaccuuguguuc

SEQ ID NO: 449 - RNA, 22 nt, homo sapiens
gacuauagaacuuucccccuca

SEQ ID NO: 450 - RNA, 22 nt, homo sapiens
gugagucucuaagaaaagagga

SEQ ID NO: 451 - RNA, 22 nt, homo sapiens
augcugacauauuuacuagagg

SEQ ID NO: 452 - RNA, 22 nt, homo sapiens
uggguuuacguugggagaacuu

SEQ ID NO: 453 - RNA, 23 nt, homo sapiens
aaagacauaggauagagucaccu

SEQ ID NO: 454 - RNA, 21 nt, homo sapiens
agacacauuuggagagggaac

SEQ ID NO: 455 - RNA, 22 nt, homo sapiens
acuuguaugcuagcucagguag

SEQ ID NO: 456 - RNA, 22 nt, homo sapiens
uuuaggauaagcuugacuuuug

SEQ ID NO: 457 - RNA, 21 nt, homo sapiens
aauggcgccacuaggguugug

SEQ ID NO: 458 - RNA, 21 nt, homo sapiens
uucacuggaguuuguuucaau

SEQ ID NO: 459 - RNA, 20 nt, homo sapiens
uaugucugcugaccaucacc

SEQ ID NO: 460 - RNA, 22 nt, homo sapiens
auaauacaugguuaaccucuuu

SEQ ID NO: 461 - RNA, 21 nt, homo sapiens
aauauuauacagucaaccucu

SEQ ID NO: 462 - RNA, 22 nt, homo sapiens
aggaccuucccugaaccaagga

SEQ ID NO: 463 - RNA, 23 nt, homo sapiens
uacccauugcauaucggaguugu

SEQ ID NO: 464 - RNA, 22 nt, homo sapiens
accaggaggcugaggccccuca

SEQ ID NO: 465 - RNA, 21 nt, homo sapiens
ugucacucggcucggcccacu

SEQ ID NO: 466 - RNA, 23 nt, homo sapiens
uuuccucauauucauucaggagu

SEQ ID NO: 467 - RNA, 24 nt, homo sapiens
aggaagcccuggaggggcuggagg

SEQ ID NO: 468 - RNA, 22 nt, homo sapiens
cuguaugcccucaccgcucagc

SEQ ID NO: 469 - RNA, 21 nt, homo sapiens
cuguccuaagguuguugaguu

SEQ ID NO: 470 - RNA, 24 nt, homo sapiens
uggaagacuagugauuuuguuguu

SEQ ID NO: 471 - RNA, 22 nt, homo sapiens
aaggagcuuacaaucuagcugg

SEQ ID NO: 472 - RNA, 22 nt, homo sapiens
ugcggggcuagggcuaacagca

SEQ ID NO: 473 - RNA, 21 nt, homo sapiens
uuugugaccugguccacuaac

SEQ ID NO: 474 - RNA, 20 nt, homo sapiens
cggcucugggucugugggga

SEQ ID NO: 475 - RNA, 22 nt, homo sapiens
acuccagccccacagccucagc

SEQ ID NO: 476 - RNA, 24 nt, homo sapiens
ugcaccaugguugucugagcaugc

SEQ ID NO: 477 - RNA, 22 nt, homo sapiens
ugagaccucuggguucugagcu

SEQ ID NO: 478 - RNA, 21 nt, homo sapiens
uccaguaccacgugucagggc

SEQ ID NO: 479 - RNA, 21 nt, homo sapiens
gcaggaacuugugagucuccu

SEQ ID NO: 480 - RNA, 22 nt, homo sapiens
cugcccuggcccgagggaccga

SEQ ID NO: 481 - RNA, 21 nt, homo sapiens
ccuggaaacacugagguugug

SEQ ID NO: 482 - RNA, 21 nt, homo sapiens
uggauuucuuugugaaucacc

SEQ ID NO: 483 - RNA, 22 nt, homo sapiens
uccauuacacuacccugccucu

SEQ ID NO: 484 - RNA, 24 nt, homo sapiens
gugaacgggcgccaucccgaggcu

SEQ ID NO: 485 - RNA, 21 nt, homo sapiens
uacucaaaaagcugucaguca

SEQ ID NO: 486 - RNA, 21 nt, homo sapiens
uuaauaucggacaaccauugu

SEQ ID NO: 487 - RNA, 21 nt, homo sapiens
uacuuggaaaggcaucaguug

SEQ ID NO: 488 - RNA, 22 nt, homo sapiens
ugcaacgaaccugagccacuga

SEQ ID NO: 489 - RNA, 22 nt, homo sapiens
ugcaacuuaccugagucauuga

SEQ ID NO: 490 - RNA, 22 nt, homo sapiens
cacuguguccuuucugcguaga

SEQ ID NO: 491 - RNA, 22 nt, homo sapiens
cacuggcuccuuucuggguaga

SEQ ID NO: 492 - RNA, 23 nt, homo sapiens
ucuuugguuaucuagcuguauga

SEQ ID NO: 493 - RNA, 22 nt, homo sapiens
uauugcacuugucccggccugu

SEQ ID NO: 494 - RNA, 22 nt, homo sapiens
uauugcacucgucccggccucc

SEQ ID NO: 495 - RNA, 23 nt, homo sapiens
caaagugcuguucgugcagguag

SEQ ID NO: 496 - RNA, 22 nt, homo sapiens
ugucuacuacuggagacacugg

SEQ ID NO: 497 - RNA, 21 nt, homo sapiens
auccgcgcucugacucucugc

SEQ ID NO: 498 - RNA, 21 nt, homo sapiens
uucucuguuuuggccaugugu

SEQ ID NO: 499 - RNA, 22 nt, homo sapiens
aaauuauuguacaucggaugag

SEQ ID NO: 500 - RNA, 21 nt, homo sapiens
uucaacggguauuuauugagc

SEQ ID NO: 501 - RNA, 23 nt, homo sapiens
uuuggcacuagcacauuuuugcu

SEQ ID NO: 502 - RNA, 22 nt, homo sapiens
ugagguaguaaguuguauuguu

SEQ ID NO: 503 - RNA, 21 nt, homo sapiens
aacccguagauccgaucuugu

SEQ ID NO: 504 - RNA, 22 nt, homo sapiens
cacccguagaaccgaccuugcg

SEQ ID NO: 505 - RNA, 21 nt, homo sapiens
uacaguacugugauaacugaa

SEQ ID NO: 506 - RNA, 22 nt, homo sapiens
acggguuaggcucuugggagcu

SEQ ID NO: 507 - RNA, 22 nt, homo sapiens
cuccuacauauuagcauuaaca

SEQ ID NO: 508 - RNA, 21 nt, homo sapiens
cuauacaaucuacugucuuuc

(19) United States
(12) Patent Application Publication
(10) Pub. No.: US 2016/0138106 A1
(43) Pub. Date: May 19, 2016

(54) Circulating Non-coding RNA Profiles for Detection of Cardiac Transplant Rejection

(71) Applicant: The Trustees of Columbia University in the City of New York, New York, NY (US)

(72) Inventors: Paul Christian Schulze, New York, NY (US); Raymond Givens, New York, NY (US)

(21) Appl. No.: 14/942,102

(22) Filed: Nov. 16, 2015

Related U.S. Application Data
(60) Provisional application No. 62/079,809, filed on Nov. 14, 2014.

(51) Int. Cl. C12Q 1/68

(52) U.S. Cl. CPC C12Q 1/6883; C12Q 2600/178; C12Q 2600/16

(57) ABSTRACT

The level of miRNAs in a sample from a patient who has received a transplant is assayed and used as an indicator for transplant rejection. Based on the measured level of the miRNAs, therapeutic intervention, such as an immunosuppressant therapy, may be started, adjusted, continued or discontinued.

### CROSS REFERENCE TO RELATED APPLICATION

This application claims priority to U.S. Provisional Application No. 62/079,809 filed on Nov. 14, 2014, which is incorporated herein by reference in its entirety.

### STATEMENT REGARDING FEDERALLY SPONSORED RESEARCH OR DEVELOPMENT

This invention was made with government support under the RO1, K23 HL095742-01, and P30 HL101272-01 grant awarded by the National Heart, Lung, and Blood Institute (NHLBI), as well as the T32 training grant awarded by the National Institutes of Health (NIH). The government may have certain rights in the invention.

### INCORPORATION-BY-REFERENCE OF SEQUENCE LISTING

A sequence listing, created on Nov. 16, 2015 as the ASCII text file “4361-0016-Seq_Listing.txt” having a file size of 75 kilobytes, is incorporated herein by reference in its entirety.

### FIELD OF THE INVENTION

The present invention relates to the detection of microRNAs for evaluating or monitoring immunological rejection after heart transplant in a patient.

### BACKGROUND OF THE INVENTION

Heart failure (HF) is associated with high morbidity as well as significant mortality. There has been an increased incidence of the disease worldwide. The clinical syndrome of heart failure is the result of heterogeneous myocardial or vascular diseases, and is defined by insufficiency to maintain blood circulation throughout the body. Despite significant advances in the clinical management of HF, conventional therapies are ultimately ineffective in many patients who progress to advanced HF. In these cases, implantation of left ventricular assist devices (LVAD) and/or heart transplantation can be the only viable options.

Heart transplantation (HTx) remains the definitive treatment for severe heart failure. The most common procedure is to take a working heart from a recently deceased organ donor (allograft) and implant it into the recipient. The recipient's heart may either be removed (orthotopic procedure), or less commonly, left in to support the donor heart (heterotopic procedure). Although less successful in comparison to allograft, it is also possible to take a heart from another species (xenograft), or implant a man-made artificial heart. U.S. Patent Application No. 20130209524.

The most common complication of heart transplant is immunological rejection which poses a significant threat to allograft function. Both acute rejection and chronic rejection can occur. Chronic rejection is the major limiting factor for the long-term success of heart transplantation. For example, growth of tissues, such as scar tissue, may cause blockage of the blood vessels of the heart, which ultimately causes the transplanted heart to fail. Two primary causes of graft failure are cell-mediated rejection (CMR) and antibody-mediated rejection (AMR).

Pharmaceutical agents such as cyclosporine A (CSA), steroids and azathioprine are used to control and suppress a recipient's immune system response to grafted tissue. Taylor et al., J. Heart Lung Transplant, 27, 943-956 (2008). Despite universal immunosuppression therapy, rejection is still the principal cause of heart transplant failures. Thus, keeping the immunological rejection to the minimum is a major objective. However, recognizing the onset and severity of rejection is difficult, while the occurrence of rejection is often unpredictable. Tissue rejection in heart transplant recipients is generally silent until the heart is damaged irreversibly. Thus, the transplanted heart tissue must be monitored continuously and carefully for signs of rejection. Early and reliable detection of graft rejection can translate into starting potentially life-saving therapy in time which is vital to the success of heart transplants. Kobashigawa, et al., J. Am. Coll. Cardiol., 45, 1532-1537 (2005).

At present, the only reliable method for monitoring and diagnosing rejection requires frequent endomyocardial biopsy (EMB), an expensive, invasive procedure that must be performed by a specialist. The biopsy is then studied by a pathologist for the invasion of heart tissue by white blood cells, edema, and dead cardiac muscle cells, the histologic manifestations of rejection. EMB is prone to sampling error; the need for repeated, invasive procedures adds significantly to cost and patient discomfort during post-transplant follow-up. Accordingly, there remains a need for a reliable, non-invasive method for detecting rejection.

MicroRNAs (miRNAs or miRs) are a class of regulatory RNAs that post-transcriptionally regulate gene expression. MiRNAs are evolutionarily conserved, small non-coding RNA molecules of approximately 18 to 25 nucleotides in length. Weiland et al., (2012) RNA Biol. 9(6):850-859. Bartel D P (2009) Cell 136(2):215-233. Each miRNA is able to downregulate hundreds of target mRNAs comprising partially complementary sequences to the miRNAs. MiRNAs act as repressors of target mRNAs by promoting their degradation, or by inhibiting translation. Braun et al. (2013) Adv. Exp. Med. Biol. 768:147-163.

MicroRNAs are promising targets for drug and biomarker development. Weiland et al. (2012) RNA Biol. 9(6):850-859. Target recognition requires base pairing of the miRNA 5′ end nucleotides (seed sequence) to complementary target mRNA regions located typically within the 3′UTR. Bartel D P (2009) Cell 136(2):215-233. Additionally, the recent detection of miRNPs (ribonucleoproteins), which contain associated miRNAs, in body fluids points towards their potential value as biomarkers for tissue injury. Laterza et al. (2009) Clin. Chem. 55:1977-1983; Ai et al. (2010) Biochem. Biophys. Res. Commun. 391:73-77. Additionally, it is also possible that miRNPs can act as paracrine and endocrine regulators of gene expression. Valadi et al. (2007) Nat. Cell Biol. 9:654-659; Williams et al. (2013) Proc. Natl. Acad. Sci. USA 110:4255-4260.

The function of miRNAs has been widely studied in animal models of HF. The muscle-specific miR-1/206 and 133a/b, and the heart-specific 208a/b, and 499, also referred to as myomirs, were shown to contribute to muscle or myocardial function. Van Rooij et al. (2007) Science 316:575-579; van Rooij et al. (2009) Dev. Cell 17:662-673. MiRNAs have been profiled in failing human myocardium, and a selected subset were also investigated as circulating biomarkers in HF (Yang et al. (2007) Nat. Med. 13:486-491; Thum et al. (2007) Circulation 116:258-267; Ikeda et al. (2007) Physiol. Genomics 31:367-373; Sucharov et al. (2008) J. Mol. Cell Cardiol. 45:185-192; Matkovich et al. (2009) Circulation 119:1263-1271; Naga et al. (2009) J. Biol. Chem. 284:27487-27499; Yang et al. (2014) Circulation 129:1009-1021; Leptidis et al. (2013) PLoS One 8:e57800; Tijsen et al. (2010) Circ. Res. 106:1035-1039; Goren et al. (2012) Eur. J. Heart Fail. 14:147-154; Dickinson et al. (2013) Eur. J. Heart Fail. 15:650-659; Corsten et al. (2010) Circ. Cardiovasc. Genet. 3:499-506; Tutarel et al. (2013) Int. J. Cardiol. 167:63-66; Fukushima et al. (2011) Circ. J. 75:336-340).

This invention describes a class of miRNAs as circulating biomarkers that allow better diagnostic assessment of patients with rejection following heart transplant or other organ/tissue transplant. The biomarkers also assist in defining the prognosis and the response to treatment.

### SUMMARY

The present invention provides for a method for diagnosing transplant rejection in a subject (e.g., human) who has received a transplant. The method may comprise the steps of: (a) obtaining a sample from the subject (e.g., a plasma, serum or blood sample, or any other sample as discussed herein); (b) assaying the level of one or more (or 2 or more, 3 or more, 4 or more, 5 or more, 6 or more, 7 or more, 8 or more, 9 or more, 10 or more, 15 or more, or 20 or more) miRNAs in the sample, wherein the one or more miRNAs are selected from Table 1 (SEQ ID NOs: 1-508); (c) comparing the level obtained in step (b) with the level of the one or more miRNAs in a control sample, and (d) diagnosing transplant rejection if the level of at least one (or at least 2, at least 3, at least 4, at least 5, at least 6, at least 7, at least 8, at least 9, or at least 10) miRNA obtained in step (b) increases or decreases by at least 5% (or at least 15%, at least 20%, at least 30%, at least 40%, at least 50%, at least 60%, at least 70%, at least 80%, at least 90%, at least 1 fold, at least 1.5 fold, at least 2 fold, at least 2.5 fold, or at least 3 fold) compared to its level in the control sample.

Also encompassed by the present invention is a method for treating a subject (e.g., human) with transplant rejection or an increased risk of transplant rejection (and/or treating a subject predicted to undergo transplant rejection). The method may comprise the steps of: (a) obtaining a sample from the subject after a transplant (e.g., a plasma, serum or blood sample, or any other sample as discussed herein); (b) assaying the level of one or more (or 2 or more, 3 or more, 4 or more, 5 or more, 6 or more, 7 or more, 8 or more, 9 or more, 10 or more, 15 or more, or 20 or more) miRNAs in the sample, wherein the one or more miRNAs are selected from Table 1 (SEQ ID NOs: 1-508); (c) comparing the level obtained in step (b) with the level of the one or more miRNAs in a control sample, and (d) treating the subject (for transplant rejection or an increased risk of transplant rejection), if the level of at least one (or at least 2, at least 3, at least 4, at least 5, at least 6, at least 7, at least 8, at least 9, or at least 10) miRNA obtained in step (b) increases or decreases by at least 5% (or at least 15%, at least 20%, at least 30%, at least 40%, at least 50%, at least 60%, at least 70%, at least 80%, at least 90%, at least 1 fold, at least 1.5 fold, at least 2 fold, at least 2.5 fold, or at least 3 fold) compared to its level in the control sample.

The transplant can be a heart transplant, a kidney transplant, a pancreas transplant, a liver transplant, a lung transplant, an intestine transplant, or a combination thereof.

The miRNAs detected may be miR-1, miR-101, miR-155, miR-125b, miR-142-3p, miR-144, miR-223-3p, miR-27a, let-7a, a let-7 family member, miR-15b*, miR-23a, miR-99b, miR-126, miR-191, miR-199a-5p, miR-425*, miR-766, or combinations thereof. In one embodiment, the miRNA detected may be let-7a, miR-101, miR-144, or combinations thereof.

The subject may be treated with an immunosuppressant. The subject's existing immunosuppressive regimen may be modified or maintained.

The level of the one or more microRNAs may be determined by RNA sequencing, microarray profiling or real-time PCR.

The control sample may be from a subject who has received a transplant without rejection or from a plurality of subjects who have received a transplant without rejection.

The present invention also provides for a kit comprising: miRNA-specific primers for reverse transcribing and/or amplifying one or more (or 2 or more, 3 or more, 4 or more, 5 or more, 6 or more, 7 or more, 8 or more, 9 or more, 10 or more, 15 or more, or 20 or more) miRNAs selected from Table 1 (SEQ ID NOs: 1-508), in a plasma or serum sample from a subject who has received a transplant; and instructions for measuring the one or more miRNAs for diagnosing or predicting transplant rejection in the subject.

The miRNA-specific primers may be for miRNAs selected from miR-1, miR-101, miR-155, miR-125b, miR-142-3p, miR-144, miR-223-3p, miR-27a, let-7a, a let-7 family member, miR-15b*, miR-23a, miR-99b, miR-126, miR-191, miR-199a-5p, miR-425*, miR-766, or combinations thereof.

The kit may additionally contain a labeled-nucleic acid probe specific for each miRNA of the kit.

### BRIEF DESCRIPTION OF THE FIGURES

FIG. 1 shows the scheme of RT-PCR.

FIG. 2 shows the levels of miRNAs let-7a, miR-223-3p, miR-101 and miR-142-3p associated with HTx (without rejection) and transplant rejection.

FIG. 3 shows the levels of miRNAs miR-144, miR-27a, miR-155, miR-125b, and miR-1 associated with HTx (without rejection) and transplant rejection.

FIG. 4 shows a receiver operating characteristic (ROC) curve which suggests that a combination of let-7a, miR-101 and miR-144 can distinguish between HTx (without rejection) and transplant rejection.

### DETAILED DESCRIPTION

The methods of the present invention assay the levels of miRNAs in a plasma or serum sample taken from a patient who has received a transplant, such as a heart transplant. The levels of miRNAs in the sample can be used for assessing the onset or severity of transplant rejection, or as an indicator of the efficacy of a therapeutic intervention for treating transplant rejection. A plurality of miRNAs may be measured. Based on the levels of the miRNAs, transplant rejection may be diagnosed or predicted, and then the subject may be treated. For patients under an immunosuppressive therapy, based on the miRNA levels, the therapeutic intervention may be continued when it is effective, or altered if ineffective.

The method may also identify transplant patients at risk for transplant rejection or delayed graft function. As such, the methods of the invention can impact the way transplant recipients are treated (before, during, and/or after a transplantation procedure). For example, patients identified as having a high risk of transplant rejection can be treated more aggressively with, for example, immunosuppressants or other therapeutic agents. To the contrary, patients identified as low risk may be treated less aggressively (e.g., with minimal immunosuppressants).

The present methods can diagnose or predict transplant rejection in a subject who has received a transplant. The method may contain the following steps: (a) obtaining a sample (e.g., a plasma or serum sample, or other samples as discussed herein) from the subject; (b) assaying the level of one or more miRNAs in the sample; and (c) comparing the level obtained in step (b) with the level of the one or more miRNAs in a control sample. The subject is diagnosed to undergo transplant rejection (or predicted to undergo transplant rejection), if the level of at least one miRNA obtained in step (b) increases or decreases by at least 5% compared to its level in the control sample.

When diagnosed with transplant rejection, the subject may be treated with at least one immunosuppressant. Alternatively, when transplant rejection is predicted, the subject may be treated with at least one immunosuppressant.

The present methods may treat a subject with transplant rejection or an increased risk of transplant rejection. The method may contain the following steps: (a) obtaining a sample (e.g., a plasma or serum sample, or other samples as discussed herein) from the subject; (b) assaying the level of one or more miRNAs in the sample; (c) comparing the level obtained in step (b) with the level of the one or more miRNAs in a control sample; and (d) treating the subject for transplant rejection or an increased risk of transplant rejection, if the level of at least one miRNA obtained in step (b) increases or decreases by at least 5% compared to its level in the control sample.

The level of at least one, or at least 2 (or at least 3, at least 4, at least 5, at least 6, at least 7, at least 8, at least 9, at least 10, at least 20, at least 30, at least 40, at least 50, between 5 and 30, between 5 and 10, between 2 and 6, between 3 and 5, between 10 and 20, between 30 and 50, or between 50 and 100) miRNAs in the sample may increase or decrease by about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, at least about 5%, at least about 10%, at least about 15%, at least about 20%, at least about 30%, at least about 40%, at least about 50%, at least about 60%, at least about 70%, at least about 80%, at least about 90%, at least about 100%, about 10% to about 90%, about 12.5% to about 80%, about 20% to about 70%, about 25% to about 60%, or about 25% to about 50%, about 2 fold, about 3 fold, about 4 fold, about 5 fold, about 6 fold, about 7 fold, about 8 fold, about 9 fold, about 10 fold, at least 1.1 fold, at least 1.2 fold, at least 1.3 fold, at least 1.4 fold, at least 1.5 fold, at least 1.6 fold, at least 1.8 fold, at least 2 fold, at least 2.5 fold, at least 3 fold, at least 3.5 fold, at least 5 fold, at least 10 fold, at least 15 fold, at least 20 fold, at least 50 fold, at least 100 fold, at least 120 fold, from about 2 fold to about 500 fold, from about 1.1 fold to about 10 fold, from about 1.1 fold to about 5 fold, from about 1.5 fold to about 5 fold, from about 2 fold to about 5 fold, from about 3 fold to about 4 fold, from about 5 fold to about 10 fold, from about 5 fold to about 200 fold, from about 10 fold to about 150 fold, from about 10 fold to about 20 fold, from about 20 fold to about 150 fold, from about 20 fold to about 50 fold, from about 30 fold to about 150 fold, from about 50 fold to about 100 fold, from about 70 fold to about 150 fold, from about 100 fold to about 150 fold, from about 10 fold to about 100 fold, from about 100 fold to about 200 fold, compared to the level(s) in the control sample. The control sample may be from a patient who has received a transplant without rejection or a plurality of patients who have received a transplant without rejection. The control sample may be from a healthy subject or a plurality of healthy subjects.

In certain embodiments, the levels of a plurality of miRNAs in the sample may be assayed, which comprises 2 or more, 3 or more, 4 or more, 5 or more, 6 or more, 7 or more, 8 or more, 9 or more, 10 or more, 15 or more, 20 or more, 25 or more, 30 or more, 35 or more, 3-504, 5-504, 10-504, 15-504, 20-504, 30-504, 50-100, 100-200, 200-300, or 300-400 miRNAs listed in Table 1 (SEQ ID NOs: 1-508).

In one embodiment, the present method determines the level of one or more miRNAs selected from miR-1, miR-101, miR-155, miR-125b, miR-142-3p, miR-144, miR-223-3p, miR-27a, and let-7a. In another embodiment, the method determines the level of one or more miRNAs selected from miR-101, miR-144, miR-223-3p and let-7a. In a third embodiment, the method determines the level of one or more miRNAs selected from miR-101, miR-144, and let-7a. In a fourth embodiment, the method determines the level of one or more miRNAs selected from miR-101, miR-223-3p, and let-7a. In a fifth embodiment, the method determines the level of one or more miRNAs selected from miR-101, miR-223-3p, let-7a, and miR-142-3p. In a sixth embodiment, the method determines the level of one or more miRNAs selected from a let-7 family member, miR-15b*, miR-23a, miR-99b, miR-126, miR-191, miR-199a-5p, miR-425*, and miR-766.

In certain embodiments, the present method determines the level of one or more miRNAs that can be any combination of one or more miRNAs selected from miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126, and miR-203. The one or more miRNAs with increased or decreased levels in the sample compared to a control sample can be any combination of one or more miRNAs selected from miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320.

Also encompassed by the present invention is a method for assessing efficacy of an immunosuppressant therapy for transplant rejection in a patient. The method may contain the following steps: (a) obtaining a first sample from the patient before initiation of the therapy (or at a first time point after initiation of the therapy); (b) assaying the levels of one or more miRNAs in the first sample; (c) obtaining a second sample from the patient after initiation of the therapy (or at a second time point after initiation of the therapy); (d) assaying the levels of the one or more miRNAs in the second sample; (e) comparing the levels of step (b) with the levels of step (d). If the level of at least one, or at least 2 (at least 3, at least 4, at least 5, at least 6, at least 7, at least 8, at least 9, at least 10, at least 20, at least 30, at least 40, at least 50, between 5 and 30, between 5 and 10, between 10 and 20, between 30 and 50, or between 50 and 100) miRNAs obtained in step (d) increases or decreases by about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, about 5%, about 10%, about 20%, about 30%, about 40%, about 50%, about 60%, about 70%, about 80%, about 90%, about 100%, about 10% to about 90%, about 12.5% to about 80%, about 20% to about 70%, about 25% to about 60%, or about 25% to about 50%, about 2 fold, about 3 fold, about 4 fold, about 5 fold, about 6 fold, about 7 fold, about 8 fold, about 9 fold, about 10 fold, at least 1.1 fold, at least 1.2 fold, at least 1.3 fold, at least 1.4 fold, at least 1.5 fold, at least 1.6 fold, at least 1.8 fold, at least 2 fold, at least 5 fold, at least 10 fold, at least 15 fold, at least 20 fold, at least 50 fold, at least 100 fold, at least 120 fold, from about 2 fold to about 500 fold, from about 1.1 fold to about 10 fold, from about 1.1 fold to about 5 fold, from about 1.5 fold to about 5 fold, from about 2 fold to about 5 fold, from about 3 fold to about 4 fold, from about 5 fold to about 10 fold, from about 5 fold to about 200 fold, from about 10 fold to about 150 fold, from about 10 fold to about 20 fold, from about 20 fold to about 150 fold, from about 20 fold to about 50 fold, from about 30 fold to about 150 fold, from about 50 fold to about 100 fold, from about 70 fold to about 150 fold, from about 100 fold to about 150 fold, from about 10 fold to about 100 fold, from about 100 fold to about 200 fold, compared to its (or their) level obtained in step (b), the therapy is considered to be effective. An effective therapy may be continued, or discontinued if the patient's condition has improved and is no longer in need of treatment. An ineffective treatment may be altered or modified, or replaced with other treatment.

The present methods can include the steps of measuring the level of at least one miRNA in a sample from a patient receiving a therapeutic intervention, and comparing the measured level to a reference level or the level of at least one miRNA in a control sample. The measured level of the at least one miRNA is indicative of the therapeutic efficacy of the therapeutic intervention.

Based on the measured miRNAs levels, therapy may be continued or altered, e.g., by change of dose or dosing frequency, or by addition of other active agents, or change of therapeutic regimen altogether.

The present invention also encompasses a method of predicting or assessing the level of severity of transplant rejection in a patient. In one embodiment, the method comprises measuring the level of at least one miRNA selected from Table 1 in a biological sample from a patient; and comparing the measured level to a reference level or the level of the at least one miRNA in a control sample, wherein the measured level of the at least one miRNA is indicative of the level of severity of transplant rejection in the patient. In other embodiments, an increase or decrease (as described herein) in the level of the miRNA is indicative of the level of severity of transplant rejection in the patient.

The expression profile of the miRNAs in a patient who has received a transplant may be determined. The expression profile of the miRNAs of the patient may be compared with a reference value, where the reference value is based on a set of miRNA expression profiles a transplant recipient in unaffected individuals or with the patients before, after and during therapy. The changes in miRNA expression may be used to alter or direct therapy, including, but not limited to, initiating, altering or stopping therapy.

Another aspect of the invention is a kit containing a reagent for measuring at least one miRNA in a biological sample, instructions for measuring at least one miRNA and instructions for evaluating or monitoring transplant rejection in a patient based on the level of the at least one miRNA. In some embodiments, the kit contains reagents for measuring from 1 to about 20 human miRNAs, including 1, 2, 3, 4, 5, 6, 7, 8, 9, 10, 11, 12, 13, 14, 15, 16, 17, 18, 19, 20 up to n from Table 1. Also encompassed by the invention are kits for assessing or predicting the severity or progression of transplant rejection in a subject. The kit may comprise a reagent for measuring at least one miRNA in a biological sample and instructions for assessing severity or progression of transplant rejection based on the level of the at least one miRNA.

TABLE 1
miRNA Sequences
The term ″hsa″ leading each miRNA name indicates
that the miRNA is a human sequence.

| SEQ | | |
|---|---|---|
| ID | | Mature miRNA |
| NO: | miRNA | Sequence (5′ to 3′) |
| 1 | hsa-let-7a | UGAGGUAGUAGGUUGUAUAGUU |
| 2 | hsa-let-7b | UGAGGUAGUAGGUUGUGUGGUU |
| 3 | hsa-let-7c | UGAGGUAGUAGGUUGUAUGGUU |
| 4 | hsa-let-7d | AGAGGUAGUAGGUUGCAUAGUU |
| 5 | hsa-let-7e | UGAGGUAGGAGGUUGUAUAGUU |
| 6 | hsa-let-7f | UGAGGUAGUAGGUUGUAUGGUU |
| 7 | hsa-let-7g | UGAGGUAGUAGUUUGUACAGUU |
| 8 | hsa-let-7i | UGAGGUAGUAGUUUGUGCUGUU |
| 9 | hsa-miR-1 | UGGAAUGUAAAGAAGUAUGUAU |
| 10 | hsa-miR-100 | AACCCGUAGAUCCGAACUUGUG |
| 11 | hsa-miR-101 | UACAGUACUGUGAUAACUGAAG |
| 12 | hsa-miR-103 | AGCAGCAUUGUACAGGGCUAUGA |
| 13 | hsa-miR-105 | UCAAAUGCUCAGACUCCUGUGGU |
| 14 | hsa-miR-106a | AAAAGUGCUUACAGUGCAGGUAG |
| 15 | hsa-miR-106b | UAAAGUGCUGACAGUGCAGAU |
| 16 | hsa-miR-107 | AGCAGCAUUGUACAGGGCUAU |
| 17 | hsa-miR-10a | UACCCUGUAGAUCCGAAUUUGU |
| 18 | hsa-miR-10b | UACCCUGUAGAACCGAAUUUGU |
| 19 | hsa-miR-1179 | AAGCAUUCUUUCAUUGGUUGGU |
| 20 | hsa-miR-1180 | UUUCCGGCUCGCGUGGGUGUGU |
| 21 | hsa-miR-1185-5p | AGAGGAUACCCUUUGUAUGUUC |
| 22 | hsa-miR-1193-5p | GGGAUGGUAGACCGGUGACGUGC |
| 23 | hsa-miR-1197 | UAGGACACAUGGUCUACUUCU |
| 24 | hsa-miR-122 | UGGAGUGUGACAAUGGUGUUUGU |
| 25 | hsa-miR-124 | UAAGGCACGCGGUGAAUGCCA |
| 26 | hsa-miR-1245-3p | AAGUGAUCUAAAGGCCUACAU |
| 27 | hsa-miR-1247 | ACCCGUCCCGUUCGUCCCCGGA |
| 28 | hsa-miR-1249 | ACGCCCUUCCCCCCCUUCUUCA |
| 29 | hsa-miR-1250 | ACGGUGCUGGAUGUGGCCUUU |
| 30 | hsa-miR-1251 | ACUCUAGCUGCCAAAGGCGCU |
| 31 | hsa-miR-1252 | AGAAGGAAAUUGAAUUCAUUU |
| 32 | hsa-miR-1255a-5p | AGGAUGAGCAAAGAAAGUAGAUU |
| 33 | hsa-miR-1255b | CGGAUGAGCAAAGAAAGUGGUU |
| 34 | hsa-miR-1256-3p | CUAAAGAGAAGUCAAUGCAUGA |
| 35 | hsa-miR-1258-3p | AGUUAGGAUUAGGUCGUGGAA |
| 36 | hsa-miR-125a | UCCCUGAGACCCUUUAACCUGU |
| 37 | hsa-miR-125b | UCCCUGAGACCCUAACUUGUGA |
| 38 | hsa-miR-126 | UCGUACCGUGAGUAAUAAUGCG |
| 39 | hsa-miR-1263-5p | AUGGUACCCUGGCAUACUGAGU |
| 40 | hsa-miR-1264 | CAAGUCUUAUUUGAGCACCUGU |
| 41 | hsa-miR-1266 | CCUCAGGGCUGUAGAACAGGGCU |
| 42 | hsa-miR-1269 | CUGGACUGAGCCGUGCUACUGG |
| 43 | hsa-miR-127-3p | UCGGAUCCGUCUGAGCUUGGCU |
| 44 | hsa-miR-1270 | CUGGAGAUAUGGAAGAGCUGUGU |
| 45 | hsa-miR-1271 | CUUGGCACCUAGCAAGCACUCA |
| 46 | hsa-miR-1277-3p | UACGUAGAUAUAUAUGUAUUUU |
| 47 | hsa-miR-1278 | UAGUACUGUGCAUAUCAUCUAU |
| 48 | hsa-miR-128 | UCACAGUGAACCGGUCUCUUU |
| 49 | hsa-miR-1283-5p | UCUACAAAGGAAAGCGCUUUCU |
| 50 | hsa-miR-1284-3p | GAAAGCCCAUGUUUGUAUUGGA |
| 51 | hsa-miR-1286 | UGCAGGACCAAGAUGAGCCCU |
| 52 | hsa-miR-1287 | UGCUGGAUCAGUGGUUCGAGU |
| 53 | hsa-miR-1289-1-3p | UGGAGUCCAGGAAUCUGCAUUU |
| 54 | hsa-miR-129-1-3p | AAGCCCUUACCCCAAAAAGUAU |
| 55 | hsa-miR-129-2-3p | AAGCCCUUACCCCAAAAAGCAU |
| 56 | hsa-miR-1293-5p | UCUGGGUGGUCUGGAGAUUUGU |
| 57 | hsa-miR-1294-5p | UGUGAGGUUGGCAUUGUUGUCU |
| 58 | hsa-miR-1295 | UUAGGCCGCAGAUCUGGGUGA |
| 59 | hsa-miR-1296 | UUAGGGCCCUGGCUCCAUCUCC |
| 60 | hsa-miR-1298-5p | UUCAUUCGGCUGUCCAGAUG |
| 61 | hsa-miR-1301 | UUGCAGCUGCCUGGGAGUGACUUC |
| 62 | hsa-miR-1303-3p | UUUUAGAGACGGGGUCUUGCUCU |
| 63 | hsa-miR-1304-5p | CGGUUUGAGGCUACAGUGAGAU |
| 64 | hsa-miR-1305 | UUUUCAACUCUAAUGGGAGAGA |
| 65 | hsa-miR-1306-5p | CCACCUCCCCUGCAAACGUCCA |
| 66 | hsa-miR-1307 | UCGACCGGACCUCGACCGGCU |
| 67 | hsa-miR-130a | CAGUGCAAUGUUAAAAGGGCAU |
| 68 | hsa-miR-130b-3p | CAGUGCAAUGAUGAAAGGGCAU |
| 69 | hsa-miR-132-3p | UAACAGUCUACAGCCAUGGUCG |
| 70 | hsa-miR-1323 | UCAAAACUGAGGGGCAUUUUCU |
| 71 | hsa-miR-133a | UUUGGUCCCCUUCAACCAGCUGU |
| 72 | hsa-miR-133b | UUUGGUCCCCUUCAACCAGCU |
| 73 | hsa-miR-134 | UGUGACUGGUUGACCAGAGGGG |
| 74 | hsa-miR-135a | UAUGGCUUUUUAUUCCUAUGUGA |
| 75 | hsa-miR-135b | UAUGGCUUUUCAUUCCUAUGUGA |
| 76 | hsa-miR-136-5p | ACUCCAUUUGUUUUGAUGAUGGA |
| 77 | hsa-miR-137 | UUAUUGCUUAAGAAUACGCGUAG |
| 78 | hsa-miR-138 | AGCUGGUGUUGUGAAUCAGGCCG |
| 79 | hsa-miR-139 | UCUACAGUGCACGUGUCUCCAGU |
| 80 | hsa-miR-140-3p | ACCACAGGGUAGAACCACGGAC |
| 81 | hsa-miR-141 | UAACACUGUCUGGUAAAGAUGGC |
| 82 | hsa-miR-142-3p | UGUAGUGUUUCCUACUUUAUGGA |
| 83 | hsa-miR-143 | UGAGAUGAAGCACUGUAGCUC |
| 84 | hsa-miR-144(-3p) | UACAGUAUAGAUGAUGUACU |
| 85 | hsa-miR-145 | GUCCAGUUUUCCCAGGAAUCCCU |
| 86 | hsa-miR-1468 | CUCCGUUUGCCUGUUUCGCUGA |
| 87 | hsa-miR-146a | UGAGAACUGAAUUCCAUGGGUU |
| 88 | hsa-miR-146b | UGAGAACUGAAUUCCAUAGGCU |
| 89 | hsa-miR-147 | GUGUGCGGAAAUGCUUCUGCU |
| 90 | hsa-miR-148a | UCAGUGCACUACAGAACUUUGU |
| 91 | hsa-miR-148b | UCAGUGCAUCACAGAACUUUGU |
| 92 | hsa-miR-149 | UCUGGCUCCGUGUCUUCACUCCC |
| 93 | hsa-miR-150 | UCUCCCAACCCUUGUACCAGUG |
| 94 | hsa-miR-151-5p | UCGAGGAGCUCACAGUCUAGU |
| 95 | hsa-miR-152 | UCAGUGCAUGACAGAACUUGG |
| 96 | hsa-miR-153 | UUGCAUAGUCACAAAAGUGAUC |
| 97 | hsa-miR-1537 | AAAACCGUCUAGUUACAGUUGU |
| 98 | hsa-miR-154-3p | AAUCAUACACGGUUGACCUAUU |
| 99 | hsa-miR-155 | UUAAUGCUAAUCGUGAUAGGGGU |
| 100 | hsa-miR-15a | UAGCAGCACAUAAUGGUUUGU |
| 101 | hsa-miR-15b | UAGCAGCACAUCAUGGUUUACA |
| 102 | hsa-miR-16 | UAGCAGCACGUAAAUAUUGGCG |
| 103 | hsa-miR-17 | CAAAGUGCUUACAGUGCAGGUAG |
| 104 | hsa-miR-181a | AACAUUCAACGCUGUCGGUGAGU |
| 105 | hsa-miR-181b | AACAUUCAUUGCUGUCGGUGGGU |
| 106 | hsa-miR-181c | AACAUUCAACCUGUCGGUGAGUUU |
| 107 | hsa-miR-181d | AACAUUCAUUGUUGUCGGUGGGU |
| 108 | hsa-miR-182 | UUUGGCAAUGGUAGAACUCACACU |
| 109 | hsa-miR-183 | UAUGGCACUGGUAGAAUUCACU |
| 110 | hsa-miR-184 | UGGACGGAGAACUGAUAAGGGU |
| 111 | hsa-miR-185 | UGGAGAGAAAGGCAGUUCCUGA |
| 112 | hsa-miR-186 | CAAAGAAUUCUCCUUUUGGGCU |
| 113 | hsa-miR-187 | UCGUGUCUUGUGUUGCAGCCGG |
| 114 | hsa-miR-188 | CAUCCCUUGCAUGGUGGAGGGU |
| 115 | hsa-miR-18a | UAAGGUGCAUCUAGUGCAGAUAG |
| 116 | hsa-miR-18b | UAAGGUGCAUCUAGUGCAGUU |
| 117 | hsa-miR-1908 | CGGCGGGGACGGCGAUUGGUC |
| 118 | hsa-miR-190a | UGAUAUGUUUGAUAUAUUAGGUU |
| 119 | hsa-miR-190b | UGAUAUGUUUGAUAUUGGGUUG |
| 120 | hsa-miR-191 | CAACGGAAUCCCAAAAGCAGCU |
| 121 | hsa-miR-1910 | CCAGUCCUGUGCCUGCCGCCU |
| 122 | hsa-miR-1911 | UGAGUACCGCCAUGUCUGUUGGG |
| 123 | hsa-miR-1912-5p | UGCUCAUUGCAUGGGCUGUGU |
| 124 | hsa-miR-1914-5p | CCCUGUGCCCGGCCCACUUCUGC |
| 125 | hsa-miR-192 | CUGACCUAUGAAUUGACAGCC |
| 126 | hsa-miR-193a-3p | AACUGGCCUACAAAGUCCCAGU |
| 127 | hsa-miR-193b | AACUGGCCCUCAAAGUCCCGCU |
| 128 | hsa-miR-194 | UGUAACAGCAACUCCAUGUGGA |
| 129 | hsa-miR-195 | UAGCAGCACAGAAAUAUUGGCA |
| 130 | hsa-miR-196a | UAGGUAGUUUCAUGUUGUUGGG |
| 131 | hsa-miR-196b | UAGGUAGUUUCCUGUUGUUGGG |
| 132 | hsa-miR-197 | UUCACCACCUUCUCCACCCAGC |
| 133 | hsa-miR-199a-3p | ACAGUAGUCUGCACAUUGGUU |
| 134 | hsa-miR-199b-3p | ACAGUAGUCUGCACAUUGGUU |
| 135 | hsa-miR-19a | UGUGCAAAUCUAUGCAAAACUGA |
| 136 | hsa-miR-19b | UGUGCAAAUCCAUGCAAAACUGA |
| 137 | hsa-miR-200a | UAACACUGUCUGGUAACGAUGUU |
| 138 | hsa-miR-200b | UAAUACUGCCUGGUAAUGAUGA |
| 139 | hsa-miR-200c | UAAUACUGCCGGGUAAUGAUGGA |
| 140 | hsa-miR-202-3p | AGAGGUAUAGGGCAUGGGAA |
| 141 | hsa-miR-203 | GUGAAAUGUUUAGGACCACUAG |
| 142 | hsa-miR-204 | UUCCCUUUGUCAUCCUAUGCCU |
| 143 | hsa-miR-205 | UCCUUCAUUCCACCGGAGUCUGU |
| 144 | hsa-miR-206 | UGGAAUGUAAGGAAGUGUGUGG |
| 145 | hsa-miR-208a | AUAAGACGAGCAAAAAGCUUGU |
| 146 | hsa-miR-208b | AUAAGACGAACAAAAGGUUUGU |
| 147 | hsa-miR-20a | UAAAGUGCUUAUAGUGCAGGUAG |
| 148 | hsa-miR-20b | CAAAGUGCUCAUAGUGCAGGUAG |
| 149 | hsa-miR-21 | UAGCUUAUCAGACUGAUGUUGAC |
| 150 | hsa-miR-210 | CUGUGCGUGUGACAGCGGCUGA |
| 151 | hsa-miR-211 | UUCCCUUUGUCAUCCUUCGCCU |
| 152 | hsa-miR-2110 | UUGGGGAAACGGCCGCUGAGUGA |
| 153 | hsa-miR-2114 | UAGUCCCUUCCUUGAAGCGGUC |
| 154 | hsa-miR-2115 | AGCUUCCAUGACUCCUGAUGGA |
| 155 | hsa-miR-2116-3p | UCCUCCCAUGCCAAGAACUCC |
| 156 | hsa-miR-212-5p | ACCUUGGCUCUAGACUGCUUACU |
| 157 | hsa-miR-214-5p | UGCCUGUCUACACUUGCUGUGC |
| 158 | hsa-miR-215 | AUGACCUAUGAAUUGACAGACA |
| 159 | hsa-miR-216a | UAAUCUCAGCUGGCAACUGUGA |
| 160 | hsa-miR-216b | AAAUCUCUGCAGGCAAAUGUGA |
| 161 | hsa-miR-217 | UACUGCAUCAGGAACUGAUUGGA |
| 162 | hsa-miR-218 | UUGUGCUUGAUCUAACCAUGU |
| 163 | hsa-miR-219-1-5p | UGAUUGUCCAAACGCAAUUCU |
| 164 | hsa-miR-219-2-3p | AGAAUUGUGGCUGGACAUCUGU |
| 165 | hsa-miR-22 | AAGCUGCCAGUUGAAGAACUGU |
| 166 | hsa-miR-221 | AGCUACAUUGUCUGCUGGGUUU |
| 167 | hsa-miR-222 | AGCUACAUCUGGCUACUGGGUCU |
| 168 | hsa-miR-223-3p | UGUCAGUUUGUCAAAUACCCCA |
| 169 | hsa-miR-224 | CAAGUCACUAGUGGUUCCGUUU |
| 170 | hsa-miR-2276-5p | GCCCUCUGUCACCUUGCAGACG |
| 171 | hsa-miR-2277 | AGCGCGGGCUGAGCGCUGCCAGU |
| 172 | hsa-miR-2278 | GAGAGCAGUGUGUGUUGCCUGG |
| 173 | hsa-miR-2355-5p | AUCCCCAGAUACAAUGGACAAU |
| 174 | hsa-miR-23a | AUCACAUUGCCAGGGAUUUCCA |
| 175 | hsa-miR-23b | AUCACAUUGCCAGGGAUUACC |
| 176 | hsa-miR-24 | UGGCUCAGUUCAGCAGGAACAG |
| 177 | hsa-miR-25 | CAUUGCACUUGUCUCGGUCUGA |
| 178 | hsa-miR-26a | UUCAAGUAAUCCAGGAUAGGCU |
| 179 | hsa-miR-26b | UUCAAGUAAUUCAGGAUAGGUU |
| 180 | hsa-miR-27a(-3p) | UUCACAGUGGCUAAGUUCCGC |
| 181 | hsa-miR-27b | UUCACAGUGGCUAAGUUCUGC |
| 182 | hsa-miR-28-5p | AAGGAGCUCACAGUCUAUUGAG |
| 183 | hsa-miR-296-3p | GAGGGUUGGGUGGAGGCUCUCC |
| 184 | hsa-miR-299-5p | UGGUUUACCGUCCCACAUACAU |
| 185 | hsa-miR-29a | UAGCACCAUCUGAAAUCGGUUA |
| 186 | hsa-miR-29b | UAGCACCAUUUGAAAUCAGUGUU |
| 187 | hsa-miR-29c | UAGCACCAUUUGAAAUCGGUU |
| 188 | hsa-miR-301a | CAGUGCAAUAGUAUUGUCAAAGC |
| 189 | hsa-miR-301b | CAGUGCAAUGAUAUUGUCAAAGC |
| 190 | hsa-miR-302a-5p | UAAACGUGGAUGUACUUGCUUU |
| 191 | hsa-miR-302b | UAAGUGCUUCCAUGUUUUAGUAG |
| 192 | hsa-miR-302c | AAGUGCUUCCAUGUUUCAGUGG |
| 193 | hsa-miR-302d | UAAGUGCUUCCAUGUUUGAGUGU |
| 194 | hsa-miR-3065-5p | UCAACAAAAUCACUGAUGCUGGA |
| 195 | hsa-miR-3074-5p | GUUCCUGCUGAACUGAGCCAGU |
| 196 | hsa-miR-30a | UGUAAACAUCCUCGACUGGAAGCU |
| 197 | hsa-miR-30b | UGUAAACAUCCUACACUCAGCU |
| 198 | hsa-miR-30c | UGUAAACAUCCUACACUCUCAGCU |
| 199 | hsa-miR-30d | UGUAAACAUCCCCGACUGGAAGCU |
| 200 | hsa-miR-30e | UGUAAACAUCCUUGACUGGAAGCU |
| 201 | hsa-miR-31-3p | UGCUAUGCCAACAUAUUGCCAUC |
| 202 | hsa-miR-3115 | AUAUGGGUUUACUAGUUGGU |
| 203 | hsa-miR-3117 | AUAGGACUCAUAUAGUGCCAGG |
| 204 | hsa-miR-3120 | CACAGCAAGUGUAGACAGGCA |
| 205 | hsa-miR-3124 | UUCGCGGGCGAAGGCAAAGUC |
| 206 | hsa-miR-3126-5p | UGAGGGACAGAUGCCAGAAGCA |
| 207 | hsa-miR-3127-5p | AUCAGGGCUUGUGGAAUGGGAAG |
| 208 | hsa-miR-3129-3p | AAACUAAUCUCUACACUGCUGC |
| 209 | hsa-miR-3130-3p | GCUGCACCGGAGACUGGGUAA |
| 210 | hsa-miR-3136 | CUGACUGAAUAGGUAGGGUCAU |
| 211 | hsa-miR-3138-3p | ACAGUGAGGUAGAGGGAGUGC |
| 212 | hsa-miR-3139 | UAGGAGCUCAACAGAUGCCUGUU |
| 213 | hsa-miR-3140-3p | AGCUUUUGGGAAUUCAGGUAG |
| 214 | hsa-miR-3143-5p | AUAACAUUGUAAAGCGCUUCUU |
| 215 | hsa-miR-3144 | AUAUACCUGUUCGGUCUCUUU |
| 216 | hsa-miR-3145-5p | AACUCCAAACACUCAAAACUCA |
| 217 | hsa-miR-3146-3p | CAUGCUAGGAUAGAAAGAAUGGG |
| 218 | hsa-miR-3149 | UUUGUAUGGAUAUGUGUGUGUAU |
| 219 | hsa-miR-3150-5p | CAACCUCGACGAUCUCCUCAGC |
| 220 | hsa-miR-3151 | GGUGGGGCAAUGGGAUCAGGU |
| 221 | hsa-miR-3152 | AUUGCCUCUGUUCUAACACAAG |
| 222 | hsa-miR-3155-5p | CCUCCCACUGCAGAGCCUGGGG |
| 223 | hsa-miR-3157-5p | UUCAGCCAGGCUAGUGCAGUCU |
| 224 | hsa-miR-3158 | AAGGGCUUCCUCUCUGCAGGAC |
| 225 | hsa-miR-3170 | CUGGGGUUCUGAGACAGACAGU |
| 226 | hsa-miR-3171-3p | UAUAUAGAUUCCAUAAAUCUAU |
| 227 | hsa-miR-3173 | UGCCCUGCCUGUUUUCUCCUUU |
| 228 | hsa-miR-3174 | UAGUGAGUUAGAGAUGCAGAGC |
| 229 | hsa-miR-3175 | CGGGGAGAGAACGCAGUGACGU |
| 230 | hsa-miR-3176 | ACUGGCCUGGGACUACCGGGG |
| 231 | hsa-miR-3177 | UGCACGGCACUGGGGACACGU |
| 232 | hsa-miR-3183 | GCCUCUCUCGGAGUCGCUCGGA |
| 233 | hsa-miR-3187 | UUGGCCAUGGGGCUGCGCGG |
| 234 | hsa-miR-3189 | CCCUUGGGUCUGAUGGGGUAGC |
| 235 | hsa-miR-3193 | UCCUGCGUAGGAUCUGAGGAGU |
| 236 | hsa-miR-3194-5p | GGCCAGCCACCAGGAGGGCUGC |
| 237 | hsa-miR-3198 | GUGGAGUCCUGGGGAAUGGAGA |
| 238 | hsa-miR-32 | UAUUGCACAUUACUAAGUUGC |
| 239 | hsa-miR-320-RNASEN | AAAAGCUGGGUUGAGAGGGCGA |
| 240 | hsa-miR-3200 | CACCUUGCGCUACUCAGGUCUGC |
| 241 | hsa-miR-323a | GCACAUUACACGGUCGACCUCU |
| 242 | hsa-miR-323b | CCCAAUACACGGUCGACCUCU |
| 243 | hsa-miR-324 | CGCAUCCCCUAGGGCAUUGGUGU |
| 244 | hsa-miR-325 | UUUAUUGAGGACCUCCUAUCAA |
| 245 | hsa-miR-326 | CCUCUGGGCCCUUCCUCCAG |
| 246 | hsa-miR-328 | CUGGCCCUCUCUGCCCUUCCGU |
| 247 | hsa-miR-329 | AACACACCUGGUUAACCUCUUU |
| 248 | hsa-miR-330-3p | GCAAAGCACACGGCCUGCAGAGA |
| 249 | hsa-miR-331 | GCCCCUGGGCCUAUCCUAGA |
| 250 | hsa-miR-335-5p | UCAAGAGCAAUAACGAAAAAUG |
| 251 | hsa-miR-337 | UCCUAUAUGAUGCCUUUCUUC |
| 252 | hsa-miR-338-3p | UCCAGCAUCAGUGAUUUUGUU |
| 253 | hsa-miR-339-5p | UCCCUGUCCUCCAGGAGCUCACG |
| 254 | hsa-miR-33a | GUGCAUUGUAGUUGCAUUGC |
| 255 | hsa-miR-33b | GUGCAUUGCUGUUGCAUUGC |
| 256 | hsa-miR-340 | UUAUAAAGCAAUGAGACUGAUU |
| 257 | hsa-miR-342 | UCUCACACAGAAAUCGCACCCGU |
| 258 | hsa-miR-345 | GCUGACUCCUAGUCCAGGGCU |
| 259 | hsa-miR-346 | UGUCUGCCCGCAUGCCUGCCUCU |
| 260 | hsa-miR-34a | UGGCAGUGUCUUAGCUGGUUGU |
| 261 | hsa-miR-34b | AGGCAGUGUCAUUAGCUGAUUGU |
| 262 | hsa-miR-34c | AGGCAGUGUAGUUAGCUGAUUGC |
| 263 | hsa-miR-3605-3p | CCUCCGUGUUACCUGUCCUCU |
| 264 | hsa-miR-361-5p | UUAUCAGAAUCUCCAGGGGUAC |
| 265 | hsa-miR-3611 | UUGUGAAGAAAGAAAUUCUU |
| 266 | hsa-miR-3612 | AGGAGGCAUCUUGAGAAAUGGA |
| 267 | hsa-miR-3613 | UGUUGUACUUUUUUUUUUGUUC |
| 268 | hsa-miR-3614-3p | UAGCCUUCAGAUCUUGGUGUUU |
| 269 | hsa-miR-3617 | AAAGACAUAGUUGCAAGAUGGG |
| 270 | hsa-miR-3619-5p | UCAGCAGGCAGGCUGGUGCAG |
| 271 | hsa-miR-362-5p | AAUCCUUGGAACCUAGGUGUGAGU |
| 272 | hsa-miR-3622-5p | CAGGCACGGGAGCUCAGGUGAG |
| 273 | hsa-miR-363 | AAUUGCACGGUAUCCAUCUGUA |
| 274 | hsa-miR-365 | UAAUGCCCCUAAAAAUCCUUAU |
| 275 | hsa-miR-3657 | UUGUGUCCCAUUAUUGGUGAUU |
| 276 | hsa-miR-3659 | UGAGUGUUGUCUACGAGGGCAU |
| 277 | hsa-miR-3664-5p | AACUCUGUCUUCACUCAUGAGU |
| 278 | hsa-miR-3667-3p | ACCUUCCUCUCCAUGGGUCUUU |
| 279 | hsa-miR-367 | AAUUGCACUUUAGCAAUGGUGA |
| 280 | hsa-miR-3677-3p | CUCGUGGGCUCUGGCCACGGCC |
| 281 | hsa-miR-3679-5p | UGAGGAUAUGGCAGGGAAG |
| 282 | hsa-miR-3680 | ACUCACUCACAGGAUUGUGCA |
| 283 | hsa-miR-3681 | UAGUGGAUGAUGCACUCUGUGC |
| 284 | hsa-miR-3682-3p | UGAUGAUACAGGUGGAGGUAG |
| 285 | hsa-miR-3688 | UAUGGAAAGACUUUGCCACUCU |
| 286 | hsa-miR-369 | AAUAAUACAUGGUUGAUCUUU |
| 287 | hsa-miR-3691 | UAGUGGAUGAUGGAGACUCGGU |
| 288 | hsa-miR-370 | GCCUGCUGGGGUGGAACCUGGU |
| 289 | hsa-miR-371 | ACUCAAACUGUGGGGGCACUU |
| 290 | hsa-miR-372 | AAAGUGCUGCGACAUUUGAGCGU |
| 291 | hsa-miR-373 | GAAGUGCUUCGAUUUUGGGGUGU |
| 292 | hsa-miR-374a | UUAUAAUACAACCUGAUAAGUG |
| 293 | hsa-miR-374b | AUAUAAUACAACCUGCUAAGUG |
| 294 | hsa-miR-375 | UUUGUUCGUUCGGCUCGCGUGA |
| 295 | hsa-miR-376a-3p | AUCAUAGAGGAAAAUCCACGU |
| 296 | hsa-miR-376b | AUCAUAGAGGAAAAUCCAUGU |
| 297 | hsa-miR-376c | AACAUAGAGGAAAUUCCACGU |
| 298 | hsa-miR-377 | AUCACACAAAGGCAACUUUUGU |
| 299 | hsa-miR-378 | ACUGGACUUGGAGUCAGAAGGC |
| 300 | hsa-miR-379 | UGGUAGACUAUGGAACGUAGG |
| 301 | hsa-miR-380-3p | UAUGUAAUAUGGUCCACAUCU |
| 302 | hsa-miR-381 | UAUACAAGGGCAAGCUCUCUGU |
| 303 | hsa-miR-382-5p | GAAGUUGUUCGUGGUGGAUUCG |
| 304 | hsa-miR-383 | AGAUCAGAAGGUGAUUGUGGCU |
| 305 | hsa-miR-384-3p | AUUCCUAGAAAUUGUUCAUAAU |
| 306 | hsa-miR-3909 | UGUCCUCUAGGGCCUGCAGUCU |
| 307 | hsa-miR-3910 | AAAAGGCAUAAAACCAAGACA |
| 308 | hsa-miR-3912 | UAACGCAUAAUAUGGACAUGU |
| 309 | hsa-miR-3919-5p | UACUGAGUCCUUUGUUCUCUAC |
| 310 | hsa-miR-3922 | UGUGGGACUUCUGGCCUUGACU |
| 311 | hsa-miR-3928 | GGAGGAACCUUGGAGCUUCGGC |
| 312 | hsa-miR-3934 | UCAGGUGUGGAAACUGAGGCAG |
| 313 | hsa-miR-3938 | AAUUCCCUUGUAGAUAACCCGG |
| 314 | hsa-miR-3939-3p | UACGCGCAGACCACAGGAUGUC |
| 315 | hsa-miR-3940 | CAGCCCGGAUCCCAGCCCACU |
| 316 | hsa-miR-3942-5p | AGCAAUACUGUUACCUGAAAU |
| 317 | hsa-miR-3944-5p | UGUGCAGCAGGCCAACCGAGA |
| 318 | hsa-miR-409-3p | GAAUGUUGCUCGGUGAACCCCU |
| 319 | hsa-miR-410 | AAUAUAACACAGAUGGCCUGU |
| 320 | hsa-miR-411 | AUAGUAGACCGUAUAGCGUACG |
| 321 | hsa-miR-412-3p | UUCACCUGGUCCACUAGCCG |
| 322 | hsa-miR-421 | AUCAACAGACAUUAAUUGGGCGC |
| 323 | hsa-miR-423-3p | AGCUCGGUCUGAGGCCCCUCAGU |
| 324 | hsa-miR-424 | CAGCAGCAAUUCAUGUUUUGA |
| 325 | hsa-miR-425 | AAUGACACGAUCACUCCCGUUGAGU |
| 326 | hsa-miR-429 | UAAUACUGUCUGGUAAAACCGU |
| 327 | hsa-miR-431-5p | UGUCUUGCAGGCCGUCAUGCA |
| 328 | hsa-miR-432 | UCUUGGAGUAGGUCAUUGGGUGG |
| 329 | hsa-miR-4326 | UGUUCCUCUGUCUCCCAGACUCU |
| 330 | hsa-miR-433 | AUCAUGAUGGGCUCCUCGGUGU |
| 331 | hsa-miR-448 | UUGCAUAUGUAGGAUGUCCCA |
| 332 | hsa-miR-449a | UGGCAGUGUAUUGUUAGCUGGU |
| 333 | hsa-miR-449b | AGGCAGUGUAUUGUUAGCUGGCU |
| 334 | hsa-miR-449c-3p | CAGUUGCUAGUUGCACUCCUCU |
| 335 | hsa-miR-450a | UUUUGCGAUGUGUUCCUAAUAU |
| 336 | hsa-miR-450b | UUUUGCAAUAUGUUCCUGAAUA |
| 337 | hsa-miR-451-DICER1 | AAACCGUUACCAUUACUGA |
| 338 | hsa-miR-452 | AACUGUUUGCAGAGGAAACUGA |
| 339 | hsa-miR-454 | UAGUGCAAUAUUGCUUAUAGGGU |
| 340 | hsa-miR-455-5p | UAUGUGCCUUUGGACUACAUCG |
| 341 | hsa-miR-466 | UGUGUUGCAUGUGUGUAUAUGU |
| 342 | hsa-miR-483-3p | CACUCCUCUCCUCCCGUCUUCU |
| 343 | hsa-miR-484*- | CCGGGGGGGGCGGGGCCUCGCG |
| | RNASEN | |
| 344 | hsa-miR-485-3p | GUCAUACACGGCUCUCCUCUCU |
| 345 | hsa-miR-486 | UCCUGUACUGAGCUGCCCCGAG |
| 346 | hsa-miR-487a-3p | AAUCAUACAGGGACAUCCAGUU |
| 347 | hsa-miR-487b | AAUCGUACAGGGUCAUCCACUU |
| 348 | hsa-miR-488 | UUGAAAGGCUAUUUCUUGGUC |
| 349 | hsa-miR-489 | GUGACAUCACAUAUACGGCAGC |
| 350 | hsa-miR-490-5p | CCAUGGAUCUCCAGGUGGGU |
| 351 | hsa-miR-491 | AGUGGGGAACCCUUCCAUGAGGA |
| 352 | hsa-miR-493 | UUGUACAUGGUAGGCUUUCAUU |
| 353 | hsa-miR-494 | UGAAACAUACACGGGAAACCUCU |
| 354 | hsa-miR-495 | AAACAAACAUGGUGCACUUCUU |
| 355 | hsa-miR-496* | GGUUGUCCAUGGUGUGUUCAUU |
| 356 | hsa-miR-497 | CAGCAGCACACUGUGGUUUGU |
| 357 | hsa-miR-498-5p | UUUCAAGCCAGGGGGCGUUUUUC |
| 358 | hsa-miR-499 | UUAAGACUUGCAGUGAUGUUU |
| 359 | hsa-miR-500a | AUGCACCUGGGCAAGGAUUCUGA |
| 360 | hsa-miR-500b | UAAUCCUUGCUACCUGGGUGAGA |
| 361 | hsa-miR-501-3p | AAUGCACCCGGGCAAGGAUUCU |
| 362 | hsa-miR-502 | AAUGCACCUGGGCAAGGAUUCA |
| 363 | hsa-miR-503 | UAGCAGCGGGAACAGUUCUGCAG |
| 364 | hsa-miR-504 | GACCCUGGUCUGCACUCUAUC |
| 365 | hsa-miR-505 | CGUCAACACUUGCUGGUUUCCU |
| 366 | hsa-miR-506 | GUAAGGCACCCUUCUGAGUAGA |
| 367 | hsa-miR-508 | UGAUUGUAGCCUUUUGGAGUAGA |
| 368 | hsa-miR-509-3p | UGAUUGGUACGUCUGUGGGUAGA |
| 369 | hsa-miR-510-3p | UGAUUGAAACCUCUAAGAGUGGA |
| 370 | hsa-miR-511-5p | GUGUCUUUUGCUCUGCAGUCA |
| 371 | hsa-miR-512-3p | AAGUGCUGUCAUAGCUGAGGUC |
| 372 | hsa-miR-513a-3p | UAAAUUUCACCUUUCUGAGAAGG |
| 373 | hsa-miR-513b | UUCACAAGGAGGUGUCAUUUAU |
| 374 | hsa-miR-513c-5p | UUCUCAAGGAGGUGUCGUUUAU |
| 375 | hsa-miR-514a | AUUGACACUUCUGUGAGUAGA |
| 376 | hsa-miR-514b-5p | UUCUCAAGAGGGAGGCAAUCAU |
| 377 | hsa-miR-515-3p | GAGUGCCUUCUUUUGGAGCGUU |
| 378 | hsa-miR-516a | UUCUCGAGGAAAGAAGCACUUU |
| 379 | hsa-miR-516b-1 | AUCUGGAGGUAAGAAGCACUUUCU |
| 380 | hsa-miR-516b-2 | AUCUGGAGGUAAGAAGCACUUU |
| 381 | hsa-miR-517a | AUCGUGCAUCCCUUUAGAGUGU |
| 382 | hsa-miR-517b | AUCGUGCAUCCUUUUAGAGUGU |
| 383 | hsa-miR-518a-3p | GAAAGCGCUUCCCUUUGCUGGA |
| 384 | hsa-miR-518b | CAAAGCGCUCCCCUUUAGAGGU |
| 385 | hsa-miR-518c | CAAAGCGCUUCUCUUUAGAGUGU |
| 386 | hsa-miR-518d | CAAAGCGCUUCCCUUUGGAGCG |
| 387 | hsa-miR-518e-3p | AAAGCGCUUCCCUUCAGAGUGU |
| 388 | hsa-miR-518f | GAAAGCGCUUCUCUUUAGAGGA |
| 389 | hsa-miR-519a | AAAGUGCAUCCUUUUAGAGUGU |
| 390 | hsa-miR-519b | AAAGUGCAUCCUUUUAGAGGUU |
| 391 | hsa-miR-519c | AAAGUGCAUCUUUUUAGAGGAU |
| 392 | hsa-miR-519d | CAAAGUGCCUCCCUUUAGAGUGU |
| 393 | hsa-miR-519e-5p | UUCUCCAAAAGGGAGCACUUUC |
| 394 | hsa-miR-520a | CUCCAGAGGGAAGUACUUUCU |
| 395 | hsa-miR-520b-3p | AAAGUGCUUCCUUUUAGAGGGU |
| 396 | hsa-miR-520c | AAAGUGCUUCCUUUUAGAGGGU |
| 397 | hsa-miR-520d-3p | AAAGUGCUUCUCUUUGGUGGGU |
| 398 | hsa-miR-520e | AAAGUGCUUCCUUUUUGAGGGU |
| 399 | hsa-miR-520f | CAAGUGCUUCCUUUUAGAGGGU |
| 400 | hsa-miR-520g | ACAAAGUGCUUCCCUUUAGAGUGU |
| 401 | hsa-miR-520h | AAAGUGCUUCCCUUUAGAGUUA |
| 402 | hsa-miR-521 | AACGCACUUCCCUUUAGAGUGU |
| 403 | hsa-miR-522 | AAAAUGGUUCCCUUUAGAGUGU |
| 404 | hsa-miR-523-3p | AACGCGCUUCCCUAUAGAGGGU |
| 405 | hsa-miR-524 | CUACAAAGGGAAGCACUUUCUC |
| 406 | hsa-miR-525-5p | CUCCAGAGGGAUGCACUUUCUC |
| 407 | hsa-miR-526a-1-3p | GAAAGCGCUUCCUUUUAGAGGA |
| 408 | hsa-miR-526a-2-3p | GAACAUGCAUCCUUUCAGAGGG |
| 409 | hsa-miR-526b-5p | CUCUUGAGGGAAGCACUUUCUGU |
| 410 | hsa-miR-527-5p | CUGCAAAGGGAAGCCCUUUCU |
| 411 | hsa-miR-532-5p | CAUGCCUUGAGUGUAGGACCGU |
| 412 | hsa-miR-539 | AUCAUACAAGGACAAUUUCUUU |
| 413 | hsa-miR-541-3p | UGGUGGGCACAGAAUCUGGACU |
| 414 | hsa-miR-542 | UGUGACAGAUUGAUAACUGAAA |
| 415 | hsa-miR-543 | AAACAUUCGCGGUGCACUUCUU |
| 416 | hsa-miR-544-5p | UCUUGUUAAAAAGCAGAUUCU |
| 417 | hsa-miR-545-5p | UCAGUAAAUGUUUAUUAGAUGA |
| 418 | hsa-miR-549-5p | AGCUCAUCCAUAGUUGUCACUG |
| 419 | hsa-miR-550-3p | UGUCUUACUCCCUCAGGCACAU |
| 420 | hsa-miR-551a | GCGACCCACUCUUGGUUUCC |
| 421 | hsa-miR-551b | GCGACCCAUACUUGGUUUCAG |
| 422 | hsa-miR-552-3p | AACAGGUGACUGGUUAGACAA |
| 423 | hsa-miR-556-5p | GAUGAGCUCAUUGUAAUAUGA |
| 424 | hsa-miR-559-3p | UUUGGUGCAUAUUUACUUUAGG |
| 425 | hsa-miR-561 | AUCAAGGAUCUUAAACUUUGCC |
| 426 | hsa-miR-570-3p | CGAAAACAGCAAUUACCUUUGC |
| 427 | hsa-miR-574-3p | CACGCUCAUGCACACACCCACA |
| 428 | hsa-miR-576-5p | AUUCUAAUUUCUCCACGUCUUU |
| 429 | hsa-miR-577 | GUAGAUAAAAUAUUGGUACCUG |
| 430 | hsa-miR-579-3p | UUCAUUUGGUAUAAACCGCGAUU |
| 431 | hsa-miR-580 | UUGAGAAUGAUGAAUCAUUAGG |
| 432 | hsa-miR-581 | UCUUGUGUUCUCUAGAUCAGU |
| 433 | hsa-miR-582 | UUACAGUUGUUCAACCAGUUACU |
| 434 | hsa-miR-584 | UUAUGGUUUGCCUGGGACUGA |
| 435 | hsa-miR-585 | UGGGCGUAUCUGUAUGCUAGGG |
| 436 | hsa-miR-588 | UUGGCCACAAUGGGUUAGAAC |
| 437 | hsa-miR-589 | UGAGAACCACGUCUGCUCUGA |
| 438 | hsa-miR-590-5p | GAGCUUAUUCAUAAAAGUGCAG |
| 439 | hsa-miR-592 | UUGUGUCAAUAUGCGAUGAUGU |
| 440 | hsa-miR-597 | UGUGUCACUCGAUGACCACUGU |
| 441 | hsa-miR-598 | UACGUCAUCGUUGUCAUCGUCA |
| 442 | hsa-miR-599 | UUUGAUAAGCUGACAUGGGACA |
| 443 | hsa-miR-605-3p | AGAAGGCACUAUGAGAUUUAGA |
| 444 | hsa-miR-610 | UGAGCUAAAUGUGUGCUGGGA |
| 445 | hsa-miR-615-3p | UCCGAGCCUGGGUCUCCCUCU |
| 446 | hsa-miR-616-5p | ACUCAAAACCCUUCAGUGACUU |
| 447 | hsa-miR-618 | AAACUCUACUUGUCCUUCUGAGU |
| 448 | hsa-miR-624-5p | UAGUACCAGUACCUUGUGUUC |
| 449 | hsa-miR-625-3p | GACUAUAGAACUUUCCCCCUCA |
| 450 | hsa-miR-627-5p | GUGAGUCUCUAAGAAAAGAGGA |
| 451 | hsa-miR-628 | AUGCUGACAUAUUUACUAGAGG |
| 452 | hsa-miR-629 | UGGGUUUACGUUGGGAGAACUU |
| 453 | hsa-miR-641 | AAAGACAUAGGAUAGAGUCACCU |
| 454 | hsa-miR-642-3p | AGACACAUUUGGAGAGGGAAC |
| 455 | hsa-miR-643 | ACUUGUAUGCUAGCUCAGGUAG |
| 456 | hsa-miR-651 | UUUAGGAUAAGCUUGACUUUUG |
| 457 | hsa-miR-652 | AAUGGCGCCACUAGGGUUGUG |
| 458 | hsa-miR-653-3p | UUCACUGGAGUUUGUUUCAAU |
| 459 | hsa-miR-654 | UAUGUCUGCUGACCAUCACC |
| 460 | hsa-miR-655 | AUAAUACAUGGUUAACCUCUUU |
| 461 | hsa-miR-656 | AAUAUUAUACAGUCAACCUCU |
| 462 | hsa-miR-659 | AGGACCUUCCCUGAACCAAGGA |
| 463 | hsa-miR-660 | UACCCAUUGCAUAUCGGAGUUGU |
| 464 | hsa-miR-665 | ACCAGGAGGCUGAGGCCCCUCA |
| 465 | hsa-miR-668 | UGUCACUCGGCUCGGCCCACU |
| 466 | hsa-miR-670 | UUUCCUCAUAUUCAUUCAGGAGU |
| 467 | hsa-miR-671 | AGGAAGCCCUGGAGGGGCUGGAGG |
| 468 | hsa-miR-675 | CUGUAUGCCCUCACCGCUCAGC |
| 469 | hsa-miR-676 | CUGUCCUAAGGUUGUUGAGUU |
| 470 | hsa-miR-7 | UGGAAGACUAGUGAUUUUGUUGUU |
| 471 | hsa-miR-708 | AAGGAGCUUACAAUCUAGCUGG |
| 472 | hsa-miR-744 | UGCGGGGCUAGGGCUAACAGCA |
| 473 | hsa-miR-758 | UUUGUGACCUGGUCCACUAAC |
| 474 | hsa-miR-760 | CGGCUCUGGGUCUGUGGGGA |
| 475 | hsa-miR-766 | ACUCCAGCCCCACAGCCUCAGC |
| 476 | hsa-miR-767 | UGCACCAUGGUUGUCUGAGCAUGC |
| 477 | hsa-miR-769 | UGAGACCUCUGGGUUCUGAGCU |
| 478 | hsa-miR-770-5p | UCAGUACCAGUGUCAGGGC |
| 479 | hsa-miR-873 | GCAGGAACUUGUGAGUCUCCU |
| 480 | hsa-miR-874 | CUGCCCUGGCCCGAGGGACCGA |
| 481 | hsa-miR-875-3p | CCUGGAAACACUGAGGUUGUG |
| 482 | hsa-miR-876-5p | UGGAUUUCUUUGUGAAUCACC |
| 483 | hsa-miR-885 | UCCAUUACACUACCCUGCCUCU |
| 484 | hsa-miR-887 | GUGAACGGGCGCCAUCCCGAGGCU |
| 485 | hsa-miR-888 | UACUCAAAAAGCUGUCAGUCA |
| 486 | hsa-miR-889 | UUAAUAUCGGACAACCAUUGU |
| 487 | hsa-miR-890-5p | UACUUGGAAAGGCAUCAGUUG |
| 488 | hsa-miR-891a | UGCAACGAACCUGAGCCACUGA |
| 489 | hsa-miR-891b | UGCAACUUACCUGAGUCAUUGA |
| 490 | hsa-miR-892a | CACUGUGUCCUUUCUGCGUAGA |
| 491 | hsa-miR-892b | CACUGGCUCCUUUCUGGGUAGA |
| 492 | hsa-miR-9 | UCUUUGGUUAUCUAGCUGUAUGA |
| 493 | hsa-miR-92a | UAUUGCACUUGUCCCGGCCUGU |
| 494 | hsa-miR-92b | UAUUGCACUCGUCCCGGCCUCC |
| 495 | hsa-miR-93 | CAAAGUGCUGUUCGUGCAGGUAG |
| 496 | hsa-miR-934 | UGUCUACUACUGGAGACACUGG |
| 497 | hsa-miR-937 | AUCCGCGCUCUGACUCUCUGC |
| 498 | hsa-miR-942 | UUCUCUGUUUUGGCCAUGUGU |
| 499 | hsa-miR-944 | AAAUUAUUGUACAUCGGAUGAG |
| 500 | hsa-miR-95 | UUCAACGGGUAUUUAUUGAGC |
| 501 | hsa-miR-96 | UUUGGCACUAGCACAUUUUUGCU |
| 502 | hsa-miR-98 | UGAGGUAGUAAGUUGUAUUGUU |
| 503 | hsa-miR-99a | AACCCGUAGAUCCGAUCUUGU |
| 504 | hsa-miR-99b | CACCCGUAGAACCGACCUUGCG |
| 505 | hsa-miR-101-3p | UACAGUACUGUGAUAACUGAA |
| 506 | hsa-miR-125b-1-3p | ACGGGUUAGGCUCUUGGGAGCU |
| 507 | hsa-miR-155-3p | CUCCUACAUAUUAGCAUUAACA |
| 508 | hsa-let-7a-3p | CUAUACAAUCUACUGUCUUUC |

miRNA

The present application measures the level of at least one miRNA in a biological sample. Samples can include any biological sample from which miRNA can be isolated. Such samples can include, but are not limited to, serum, plasma, blood, whole blood and derivatives thereof, cardiac tissue, bone marrow, urine, cerebrospinal fluid (CSF), myocardium, endothelium, skin, hair, hair follicles, saliva, oral mucus, vaginal mucus, sweat, tears, epithelial tissues, semen, seminal plasma, prostatic fluid, excreta, ascites, lymph, bile, as well as other samples or biopsies. In one embodiment, the biological sample is plasma or serum. In other embodiments, the biological sample is cardiac tissue. The miRNA may include an intron-embedded miRNA. The miRNA may be expressed in heart tissue. The miRNA may be expressed in muscles.

The sample may be obtained at any time point after the transplant procedure, such as about 10 minutes, about 30 minutes, about 1 hour, about 2 hours, about 3 hours, about 4 hours, about 5 hours, about 6 hours, about 7 hours, about 8 hours, about 10 hours, about 12 hours, about 15 hours, about 18 hours, about 20 hours, about 22 hours, about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 3 years, about 5 years or more following the transplantation procedure. The time point may also be earlier or later.

In particular embodiments, the miRNA is selected from the miRNAs listed in Table 1. In certain embodiments, the level of each microRNA in a panel of microRNAs selected from Table 1 is measured. For instance, in some embodiments, 1 or more, 2 or more, 3 or more, 4 or more, 5 or more, 10 or more, 15 or more, 20 or more, 25 or more, 30 or more, 35 or more, 40 or more, 50 or more, 60 or more, 70 or more, 80 or more, or 90 or more microRNAs selected from Table 1 are measured. In some embodiments, a panel of no greater than 20, no greater than 15, no greater than 10, or no greater than 5 miRNAs is tested, the panel including 1, 2, 3, 4, 5, 6, 7, 8, 9, 10, 11, 12, 13, 14, 15, 16, 17, 18, 19, 20 or more miRNAs from Table 1.

In one embodiment, the miRNAs detected include any of miR-1, miR-101, miR-155, miR-125b, miR-142-3p, miR-144, miR-223-3p, miR-27a, and let-7a, or any combination thereof. In another embodiment, the miRNAs detected include any of miR-101, miR-144, miR-223-3p and let-7a, or any combination thereof. In a third embodiment, the miRNAs detected include any of miR-101, miR-144, and let-7a, or any combination thereof. In a fourth embodiment, the miRNAs detected include any of miR-101, miR-223-3p, and let-7a, or any combination thereof. In a fifth embodiment, the miRNAs detected include any of miR-101, miR-223-3p, let-7a, and miR-142-3p, or any combination thereof. In a sixth embodiment, the miRNAs detected include any of a let-7 family member, miR-15b*, miR-23a, miR-99b, miR-126, miR-191, miR-199a-5p, miR-425*, and miR-766, or any combination thereof.

In another embodiment, the miRNAs detected include any of miR-208a, miR-208b, miR-499, miR-1, miR-206, miR-133a, miR-133b, miR-221, miR-216a, miR-375, miR-210, miR-1908, miR-1180, miR-195, miR-199a, miR-199b, miR-29a, miR-22, miR-122, miR-126 and miR-203 or any combination thereof. In yet another embodiment, the miRNAs detected include miR-16, miR-421, miR-195, miR-628, miR-30a, miR-30e, miR-1307, miR-142, miR-101, miR-215, miR-30a, miR-146b, miR-190a, miR-629, miR-378, miR-93, miR-106a, miR-106b, miR-15a, miR-125b, miR-199a, miR-199b, miR-100, miR-216a, miR-370, miR-766, miR-887, miR-1180, miR-129, miR-92b, miR-769, and miR-320 or any combination thereof.

The present application may also measure the level of 2, 3, 4, 5, 6 or more myomirs. As used herein, the term “myomir” may refer to any miRNA highly-enriched in cardiac and/or skeletal muscle. Myomirs may include, but are not limited to, miR-208a, miR-208b, miR-499, miR-1, miR-206, miR-133a, miR-133b, and miR-486 (McCarthy et al., 2007, MicroRNA-1 and microRNA-133a expression are decreased during skeletal muscle hypertrophy. J. Appl. Physiol. 102, 306-313; Callis et al. 2008, Exp Biol. Med (Maywood) 233, 131-138; van Rooij et al. 2008, Trends Genet 24, 159-166; van Rooij et al. 2009 Dev Cell 17, 662-673; Small et al. 2010, Proc Natl Acad Sci. 107, 4218-4223).

The level or amount of microRNA in a patient sample can be compared to a reference level or amount of the microRNA present in a control sample. The control sample may be from a patient who has received a transplant without rejection or a plurality of patients who have received a transplant without rejection. The control sample may be from a healthy subject or a plurality of healthy subjects. In other embodiments, a control sample is taken from a patient prior to treatment with a therapeutic intervention or a sample taken from an untreated patient (e.g., a patient who has not received a transplant and/or an immunosuppressant therapy). Reference levels for a microRNA can be determined by determining the level of a microRNA in a sufficiently large number of samples obtained from a patient or patients who have received a transplant without transplant rejection, or normal, healthy control subjects to obtain a pre-determined reference or threshold value. A reference level can also be determined by determining the level of the microRNA in a sample from a patient prior to treatment with the therapeutic intervention.

Reference (or calibrator) level information and methods for determining reference levels can be obtained from publically available databases, as well as other sources. (See, e.g., Bunk, D. M. (2007) Clin. Biochem. Rev., 28(4):131-137; and Remington: The Science and Practice of Pharmacy, Twenty First Edition (2005)). In some embodiments, a known quantity of an oligonucleotide or oligonucleotides (e.g., small synthetic oligonucleotides with 18-25 nucleotides; or another miRNA) that is not normally present in the sample is added to the sample (i.e., the sample is spiked with a known quantity of calibrators or exogenous oligonucleotides) and the level of one or more miRNAs of interest is calculated based on the known quantity of the spiked calibrators or oligonucleotides. In one embodiment, these spike-in calibrators have no match in the human genome and serve for quantification. In another embodiment, the abundance, level or amount of the miRNA of interest is calculated from the read ratios of the miRNA reads to spiked-in calibrator reads.

The comparison of the measured levels of the one or more miRNAs to a reference amount or the level of one or more of the miRNAs in a control sample can be done by any method known to a skilled artisan. For example, comparing the amount of the microRNA in a sample to a standard amount can include comparing the ratio between 5S rRNA (or the spiked oligonucleotides) and the miRNA in a sample to a published or known ratio between 5S rRNA (or the spiked oligonucleotides) and the miRNA in a control sample.

The level, amount, abundance or concentration of miRNAs may be measured. The measurement result may be an absolute value or may be relative (e.g., relative to a reference oligonucleotide, relative to a reference miRNA, etc.)

Measuring or detecting the amount or level of microRNA in a sample can be performed in any manner known to one skilled in the art and such techniques for measuring or detecting the level of an miRNA are well known and can be readily employed. A variety of methods for detecting miRNAs have been described and may include small RNA sequencing (sRNAseq), deep-sequencing, single-molecule direct RNA sequencing (RNAseq), Northern blotting, microarrays, real-time PCR (polymerase chain reaction), reverse transcription PCR (RT-PCR), targeted RT-PCR, in situ hybridization, miRNA Taqman array cards, electrochemical methods (e.g., oxidation of miRNA-ligated nanoparticles), bioluminescent methods, bioluminescent protein reassembly, BRET (bioluminescence resonance energy transfer)-based methods, fluorescence correlation spectroscopy and surface-enhanced Raman spectroscopy (Cissell, K. A. and Deo, S. K. (2009) Anal. Bioanal. Chem., 394:1109-1116).

The methods of the present invention may include the step of reverse transcribing RNA when assaying the level or amount of a miRNA.

Any suitable methods/kits may be used to isolate and assay the level of the miRNA. There are also commercially available kits, such as the qRT-PCR miRNA Detection Kit available from Ambion, U.S.A., which can be used for detecting and quantifying microRNA using quantitative reverse transcriptase polymerase chain reaction. TaqMan MicroRNA Assays, which employ a target-specific stern-loop reverse transcription primer to compensate for the short length of the mature miRNA, is also available from Applied Biosystems (Life Technologies, Inc., USA). qSTAR MicroRNA Detection Assays, commercially available from OriGene, Inc. (USA), can also be used. U.S. Patent Publication No. 20140024700. Other commercially available kits, such as PAXgene Blood miRNA Kit (which uses silica-based RNA purification technology) can be employed for isolating miRNAs of 18 nucleotides or longer, available from Qiagen, USA. The miScript PCR System, a three-component system which converts miRNA and mRNA into cDNA and allows for detection of miRNAs using SYBR Green-based real-time PCR, can be employed for quantification of mature miRNA, precursor miRNA, and mRNA all from a single sample (also available from Qiagen, USA). GeneCopoeia has a commercial kit available that is based on using RT-PCR in conjunction with SYBR Green for quantitation of miRNA (All-in-One™ miRNA qRT-PCR Detection Kit, available from GeneCopoeia, Inc., USA). mirVANA, available from Life Technologies, Inc. (USA), employs glass fiber filter (GFF)-based method for isolating small RNAs.

The methods for detecting miRNAs can also include hybridization-based technology platforms and massively parallel next generation small RNA sequencing that allow for detection of multiple microRNAs simultaneously. One commercially-available hybridization-based technology utilizes a sandwich hybridization assay with signal amplification provided by a labeled branched DNA (Panornics). Another hybridization-based technology is available from Nanostring Technology (nCounter miRNA Expression Assay), where multiple miRNA sequences are detected and distinguished with fluorescently-labeled sequence tags. Examples of next-generation sequencing are available from Life Technologies (SOLiD platform) and Illumina, Inc. (e.g., Illumina HumanHT-12 bead arrays).

In one embodiment, to assay miRNA levels, the reads corresponding to miRNA genes organized in miRNA cistrons may be combined. The cistrons are labeled with the corresponding miRNA name but with the “R” of “miR” in lowercase, i.e., “mir”.

MiRNAs can be isolated by methods described in the art for isolating small RNA molecules (U.S. Patent Publication No. 20100291580, U.S. Patent Publication No. 20100222564, U.S. Patent Publication No. 20060019258, U.S. Patent Publication No. 20110054009 and U.S. Patent Publication No. 20090023149).

In one embodiment, miRNA may be isolated from a sample by a method comprising the following steps: a) obtaining a sample having an miRNA; b) isolating total RNA from the sample; c) size fractionation of total RNA by, for example, gel electrophoresis (e.g., polyacrylamide gel electrophoresis) to separate RNAs of the appropriate sizes (e.g., small RNAs); d) ligating DNA adapters to one end or both ends of the separated small RNAs; e) reverse transcription of the adapter-ligated RNAs into cDNAs and PCR amplification; and (f) DNA sequencing. Steps (a)-(f) may be conducted in a different order than listed above. Any of the steps (a)-(f) may be skipped or combined.

Other methods for isolation of miRNA from a sample include employing a method comprising the following steps: a) obtaining a sample having an miRNA; b) adding an extraction solution to the sample; c) adding an alcohol solution to the extracted sample; d) applying the sample to a mineral or polymer support; and, e) eluting the RNA containing the miRNA from the mineral or polymer support with an ionic solution. Other procedures for isolating miRNA molecules from a sample can involve: a) adding an alcohol solution to the sample; b) applying the sample to a mineral or polymer solid support; c) eluting miRNA molecules from the support with an ionic solution; and, d) using or characterizing the miRNA molecules. (U.S. Patent Publication No. 20100222564).

MiRNA can also be isolated by methods involving separation of miRNA from mRNA, such as those described in U.S. Patent Publication No. 20060019258. These methods comprise the steps of a) providing a biological isolate including mRNA having a 5′ cap structure and small RNA having a 5′ phosphate; b) contacting the isolate with a phosphate reactive reagent having a label moiety under conditions wherein the label moiety is preferentially added to the 5′ phosphate over the 5′ cap structure, thereby producing labeled small RNA; and c) distinguishing the small RNA from the mRNA according to the presence of the label.

Examples of methods of isolating and/or quantifying microRNAs can also include but are not limited to hybridizing at least a portion of the microRNA with a fluorescent nucleic acid (a fluorescent probe), and reacting the hybridized microRNA with a fluorescent reagent, wherein the hybridized microRNA emits a fluorescent light or hybridizing at least a portion the microRNA to a radio-labeled complementary nucleic acid. There are commercially available products for fluorescent labeling and detection of miRNAs. NCode miRNA Rapid Labeling System and NCode Rapid Alexa Fluor 3 miRNA Labeling System are both commercially available from Life Technologies, Inc. (USA). Furthermore, fluorescent labels are commercially available and can include the Alexa Flour dyes (Molecular Probes), available from Life Technologies, Inc. (USA), Cy dyes (Lumiprobes), the DyLight fluorophores (available from ThermoScientific (USA)), and FluoProbes.

Locked nucleic acid probes can also be employed. For example, the miRCURY LNA microRNA ISH Optimization Kits (FFPE) provides for detection of microRNAs. This kit employs double DIG*-labeled miRCURY LNA™ microRNA Detection that can be used for in situ hybridization and is commercially available from Exiqon (USA and Denmark).

In one embodiment, a probe for detecting a miRNA can include a single-stranded molecule, including a single-stranded deoxyribonucleic acid molecule, a single-stranded ribonucleic acid molecule, a single-stranded peptide nucleic acid (PNA), or a single-stranded locked nucleic acid (LNA). The probe may be substantially complementary, for example 80%, 85%, 90%, 95%, 96%, 97%, 98%, 99% or 100% identical to the complement of the miRNA being detected, such that the probe is capable of detecting the miRNA. In some embodiments, the probe is substantially identical, 80%, 85%, 90%, 95%, 96%, 97%, 98%, 99%, or 100% identical to the miRNA, such that the probe is capable of detecting the complement of the miRNA. In some instances the probe is at least 5 nucleotides, at least 10 nucleotides, at least 15 nucleotides, at least 20 nucleotides, at least 25 nucleotides, at least 30 nucleotides or at least 40 nucleotides. In some cases, the probe may be no longer than 25 nucleotides, no longer than 35 nucleotides; no longer than 50 nucleotides; no longer than 75 nucleotides, no longer than 100 nucleotides or no longer than 125 nucleotides in length. In some embodiments the probe is substantially complementary to or substantially identical to at least 5 consecutive nucleotides of the miRNA, for example at least 6, 7, 8, 9, 10, 11, 12, 13, 14, 15, 16, 17, 18, 20, 21 and 22, or more consecutive nucleotides. In some embodiments, the probe can be 5-20, 5-25, 5-50, 50-100, or over 100 consecutive nucleotides long.

In one embodiment, a difference (increase or decrease) in the measured level of the miRNA relative to the level of the miRNA in the control sample (e.g., a sample in at least one patient who has received a transplant without rejection, in the patient prior to treatment, at a different time point during treatment, or an untreated patient) or a pre-determined reference value is indicative of the therapeutic efficacy of the therapeutic intervention (e.g., an immunosuppressant therapy). In another embodiment, an increase (or decrease) in the measured level of the miRNA relative to the level of the miRNA in the control sample or pre-determined reference value is indicative of the therapeutic efficacy of the therapeutic intervention. For instance, in such embodiments, when the level of one or more miRNAs selected from Table 1 is increased (or decreased) when compared to the level in a control sample or pre-determined reference value in response to a therapeutic intervention, the increase (or decrease) is indicative of therapeutic efficacy of the therapeutic intervention.

A reduction or decrease in the measured level of the miRNA relative to the level of the miRNA in the control sample (e.g., a sample in the patient prior to treatment or an untreated patient) or pre-determined reference value can be indicative of the therapeutic efficacy of the therapeutic intervention. For instance, in such embodiments, when the level of one or more miRNAs selected from Table 1 is decreased (or increased) when compared to the level in a control sample or pre-determined reference value in response to a therapeutic intervention, the decrease (or increase) is indicative of therapeutic efficacy of the therapeutic intervention.

Patients showing different (elevated or reduced) levels of miRNA, e.g., miR-1, miR-101, miR-155, miR-125b, miR-142-3p, miR-144, miR-223-3p, miR-27a, let-7a, a let-7 family member, miR-15b*, miR-23a, miR-99b, miR-126, miR-191, miR-199a-5p, miR-425*, and miR-766, or combinations (mixtures) can be identified. The expression profile of these miRNAs may be used to calculate a score for the combined or individual miRNA expression. The scores of these patients will be compared to the score of unaffected individuals (e.g., patients without transplant rejection). The clinical condition of these patients with respect to their cardiac status may be correlated with the miRNA expression profiles. The scores may be used to identify groups of patients having transplant rejection responsive to immunosuppressant treatment.

### Transplant Rejection

The present method may be used to assess the transplant status or outcome, including, but not limited to, transplant rejection, transplant function (including delayed graft function), non-rejection based allograft injury, transplant survival, chronic transplant injury, or titer pharmacological immunosuppression. In some embodiments, the non-rejection based allograft injury may include ischemic injury, virus infection, peri-operative ischemia, reperfusion injury, hypertension, physiological stress, injuries due to reactive oxygen species and/or injuries caused by pharmaceutical agents. The transplant status or outcome may comprise vascular complications or neoplastic involvement of the transplanted organ.

In some embodiments, the methods described herein are used for diagnosing or predicting transplant status or outcome (e.g., transplant rejection). In some embodiments, the methods described herein are used to detect and/or quantify target miRNAs to determine whether a subject is undergoing transplant rejection. In some embodiments, the methods described herein are used to detect and/or quantify target miRNAs for diagnosis or prediction of transplant rejection. In some embodiments, the methods described herein are used to detect and/or quantify target miRNAs for determining an immunosuppressive regimen for a subject who has received a transplant. In some embodiments, the methods described herein are used to detect and/or quantify target miRNAs to predict transplant survival in a subject that have received a transplant. The invention provides methods of diagnosing or predicting whether a transplant in a transplant recipient will survive or be lost. In certain embodiments, the methods described herein are used to detect and/or quantify target miRNAs to diagnose or predict the presence of long-term graft survival. In some embodiments, the methods described herein are used to detect and/or quantify target miRNAs for diagnosis or prediction of non-rejection based transplant injury. The present methods may be used to diagnose graft-versus-host-disease (GVHD).

As used herein the term “diagnose” or “diagnosis” of a transplant status or outcome includes predicting or diagnosing the transplant status or outcome, determining predisposition to a transplant status or outcome, monitoring treatment of transplant patient, diagnosing a therapeutic response of transplant patient, and prognosis of transplant status or outcome, transplant progression, and response to particular treatment.

The transplant may be an allograft or a xenograft. An allograft is a transplant of an organ, tissue, bodily fluid or cell from one individual to a genetically non-identical individual of the same species. A xenograft is a transplant of an organ, tissue, bodily fluid or cell from a different species.

The transplant maybe any organ or tissue transplant, including, but not limited to, a heart transplant, a kidney transplant, a liver transplant, a pancreas transplant, a lung transplant, an intestine transplant, a skin transplant, a bone marrow transplant, a small bowel transplant, a trachea transplant, a cornea transplant, a limb transplant, and a combination thereof.

The present methods may determine the presence, type and/or severity of the transplant rejection. Transplant rejection includes a partial or complete immune response to a transplanted cell, tissue, organ, or the like on or in a recipient of said transplant due to an immune response to a transplant. A transplant can be rejected through either a cell-mediated rejection (CMR) or antibody-mediated rejection (AMR). The rejection may be acute cellular rejection (ACR).

Rejection after a heart transplant may be graded according to the ISHLT (International Society for Heart and Lung Transplantation) guidelines (Table 2 and Table 3).

TABLE 2
ISHLT Standardized Cardiac Biopsy Grading
(2004): Acute Cellular Rejection (ACR)

| | |
|---|---|
| Grade 0R | No Rejection |
| Grade 1R, mild | Interstitial and/or perivascular |
| | infiltrate with up to 1 focus of |
| | myocyte damage |
| Grade 2R, moderate | Two or more foci of infiltrate with |
| | associated myocyte damage |
| Grade 3R, severe | Diffuse infiltrate with multifocal |
| | myocyte damage ± edema, ± |
| | hemorrhage ± vasculitis |

TABLE 3
ISHLT Recommendations for Acute Antibody-
Mediated Rejection (AMR) (2004)

| | |
|---|---|
| AMR 0 | Negative for acute antibody-mediated rejection |
| | No histologic or immunopathologic features of AMR |
| AMR 1 | Positive for AMR |
| | Histologic features of AMR |
| | Positive immunofluorescence or immunoperoxidase |
| | staining for AMR (positive CD68, C4d) |

The present methods may diagnose or predict any type of transplant rejection, including, but not limited to, hyperacute rejection, acute rejection, and/or chronic rejection. Hyperacute rejection can occur within minutes or hours to days following transplantation and may be mediated by a complement response in recipients with pre-existing antibodies to the donor. In hyperacute rejection, antibodies are observed in the transplant vasculature very soon after transplantation, possibly leading to clotting, ischemia, and eventual necrosis and death. Acute rejection occurs days to months or even years following transplantation. It can include a T-cell mediated response and is identified based on presence of T-cell infiltration of the transplanted tissue, structural injury to the transplanted tissue, and injury to the vasculature of the transplanted tissue. Chronic rejection occurs months to years following transplantation and is associated with chronic inflammatory and immune response against the transplanted tissue. Chronic rejection may also include chronic allograft vasculopathy, which is associated with fibrosis of vasculature of the transplanted tissue. U.S. Pat. No. 8,637,038. Fibrosis is a common factor in chronic rejection of all types of organ transplants. Chronic rejection can typically be described by a range of specific disorders that are characteristic of the particular organ. For example, in heart transplants or transplants of cardiac tissue, such as valve replacements, such disorders include fibrotic atherosclerosis; in lung transplants, such disorders include fibroproliferative destruction of the airway (bronchiolitis obliterans); in kidney transplants, such disorders include obstructive nephropathy, nephrosclerorsis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs.

In some embodiments, the invention provides methods of determining whether a patient or subject is displaying transplant tolerance. The term “transplant tolerance” includes when the subject does not reject a graft organ, tissue or cell(s) that has been introduced into/onto the subject. In other words, the subject tolerates or maintains the organ, tissue or cell(s) that has been transplanted.

Graft-versus-host-disease (GVHD) is the pathological reaction that occurs between the host and grafted tissue. The grafted or donor tissue dominates the pathological reaction. GVHD can be seen following stem cell and/or solid organ transplantation. GVHD occurs in immunocompromised subjects, who when transplanted, receive “passenger” lymphocytes in the transplanted stem cells or solid organ. These lymphocytes recognize the recipient's tissue as foreign. Thus, they attack and mount an inflammatory and destructive response in the recipient. GVHD has a predilection for epithelial tissues, especially skin, liver, and mucosa of the gastrointestinal tract. GVHD subjects are immunocompromised due the fact that prior to transplant of the graft, the subject receives immunosuppressive therapy.

Certain embodiments of the invention provide methods of predicting transplant survival in a subject that has received a transplant. The invention provides methods of diagnosing or predicting whether a transplant in a transplant patient or subject will survive or be lost. In certain embodiments, the invention provides methods of diagnosing or predicting the presence of long-term graft survival. By “long-term” graft survival is meant graft survival for at least about 5 years beyond current sampling, despite the occurrence of one or more prior episodes of acute rejection. In certain embodiments, transplant survival is determined for patients in which at least one episode of acute rejection has occurred. As such, these embodiments provide methods of determining or predicting transplant survival following acute rejection.

The level of one or more miRNAs may be assayed to diagnose or monitor other cardiac disease states including, but not limited to, diseases of the cardiac valves, other forms of cardiomyopathies, inflammatory heart disease, congenital heart disease.

### Therapeutic Intervention

Based on the levels of the miRNA(s), transplant rejection may be diagnosed or predicted, and then the subject may be treated with a therapy for the rejection, such as an immunosuppressant therapy.

An immunosuppressant, also referred to as an immunosuppressive agent, can be any compound that decreases the function or activity of one or more aspects of the immune system, such as a component of the humoral or cellular immune system or the complement system.

Non-limiting examples of immunosuppressants include, (1) antimetabolites, such as purine synthesis inhibitors (such as inosine monophosphate dehydrogenase (IMPDH) inhibitors, e.g., azathioprine, mycophenolate, and mycophenolate mofetil), pyrimidine synthesis inhibitors (e.g., leflunomide and teriflunomide), and antifolates (e.g., methotrexate); (2) calcineurin inhibitors, such as tacrolimus, cyclosporine A, pimecrolimus, and voclosporin; (3) TNF-alpha inhibitors, such as thalidomide and lenalidomide; (4) IL-1 receptor antagonists, such as anakinra; (5) mammalian target of rapamycin (mTOR) inhibitors, such as rapamycin (sirolimus), deforolimus, everolimus, temsirolimus, zotarolimus, and biolimus A9; (6) corticosteroids, such as prednisone; and (7) antibodies to any one of a number of cellular or serum targets (including anti-lymphocyte globulin and anti-thymocyte globulin).

Non-limiting exemplary cellular targets and their respective inhibitor compounds include, but are not limited to, complement component 5 (e.g., eculizumab); tumor necrosis factors (TNFs) (e.g., infliximab, adalimumab, certolizumab pegol, afelimomab and golimumab); IL-5 (e.g., mepolizumab); IgE (e.g., omalizumab); BAYX (e.g., nerelimomab); interferon (e.g., faralimomab); IL-6 (e.g., elsilimomab); IL-12 and IL-13 (e.g., lebrikizumab and ustekinumab); CD3 (e.g., muromonab-CD3, otelixizumab, teplizumab, visilizumab); CD4 (e.g., clenoliximab, keliximab and zanolimumab); CD11a (e.g., efalizumab); CD18 (e.g., erlizumab); CD20 (e.g., afutuzumab, ocrelizumab, pascolizumab); CD23 (e.g., lumiliximab); CD40 (e.g., teneliximab, toralizumab); CD62L/L-selectin (e.g., aselizumab); CD80 (e.g., galiximab); CD147/basigin (e.g., gavilimomab); CD154 (e.g., ruplizumab); BLyS (e.g., belimumab); CTLA-4 (e.g., ipilimumab, tremelimumab); CAT (e.g., bertilimumab, lerdelimumab, metelimumab); integrin (e.g., natalizumab); IL-6 receptor (e.g., tocilizumab); LFA-1 (e.g., odulimomab); and IL-2 receptor/CD25 (e.g., basiliximab, daclizumab, inolimomab).

The present invention provides for methods for evaluating and/or monitoring the efficacy of a therapeutic intervention (e.g., an immunosuppressant therapy) for treating transplant rejection. These methods can include the step of measuring the level of at least one miRNA, such as one or more miRNAs listed in Table 1, or a panel of miRNAs, in a biological sample from a patient who has received a transplant. In some embodiments, the level of the at least one miRNA in the biological sample is compared to a reference level, or the level of the at least one miRNA in a control sample. The control sample may be taken from the patient at a different time point after transplantation, or from the patient before initiation of the therapeutic intervention (e.g., an immunosuppressant therapy), or from the patient at a different time point after initiation of the therapeutic intervention (e.g., an immunosuppressant therapy). The measured level of the at least one miRNA is indicative of the therapeutic efficacy of the therapeutic intervention. In some cases, an increase or decrease in the level of the miRNA is indicative of the efficacy of the therapeutic intervention. In some embodiments, a change in the measured level of the at least one miRNA relative to a sample from the patient taken prior to treatment or earlier during the treatment regimen is indicative of the therapeutic efficacy of the therapeutic intervention.

In certain embodiments, the method comprises detecting 1, 2, 3, 4, 5, 6, 7, 8, 9, 10, 11, 12, 13, 14, 15, 16, 17, 18, 19, 20 or more miRNAs (e.g., including all miRNAs) listed in Table 1. When a panel of miRNAs is determined in the patient sample, the patient sample may be classified as indicative of effective or non-effective intervention on the basis of a classifier algorithm. For example, samples may be classified on the basis of threshold values as described, or based upon mean and/or median miRNA levels in one population or versus another (e.g., a population of healthy controls or a population of patients having received a transplant without rejection, or levels based on effective versus ineffective therapy).

Various classification schemes are known for classifying samples between two or more classes or groups, and these include, without limitation: Principal Components Analysis, Naive Bayes, Support Vector Machines, Nearest Neighbors, Decision Trees, Logistic, Artificial Neural Networks, Penalized Logistic Regression, and Rule-based schemes. In addition, the predictions from multiple models can be combined to generate an overall prediction. Thus, a classification algorithm or “class predictor” may be constructed to classify samples. The process for preparing a suitable class predictor (reviewed in Simon (2003) British Journal of Cancer (89) 1599-1604).

The present invention also provides methods for modifying a treatment regimen comprising detecting the level of at least one miRNA in a biological sample from a patient receiving the therapeutic intervention and modifying the treatment regimen based on an increase or decrease in the level of the at least one miRNA in the biological sample. The methods for modifying the treatment regimen of a therapeutic intervention may comprise the steps of: (a) detecting the level of at least one miRNA, such as one or more miRNAs listed in Table 1 in a biological sample from a patient receiving the therapeutic intervention; and (b) modifying the treatment regimen based on an increase or decrease in the level of the at least one miRNA in the biological sample. In some embodiments, the method comprises detecting 2, 3, 4, 5, 6, 7, 8, 9, 10 or more miRNAs (e.g., including all miRNAs) listed in Table 1. In some such embodiments, less than 100, less than 50, or less than 25 miRNAs are detected, including the miRNAs from Table 1.

Modifying the treatment regimen can include, but is not limited to, changing and/or modifying the type of therapeutic intervention, the dosage at which the therapeutic intervention is administered, the frequency of administration of the therapeutic intervention, the route of administration of the therapeutic intervention, as well as any other parameters that would be well known by a physician to change and/or modify. For example, where miRNAs of Table 1 decrease (or increase) during therapy or match reference levels, the therapeutic intervention is continued. In embodiments where miRNAs of Table 1 do not decrease (or increase) during therapy or match reference levels, the therapeutic intervention is modified. In another embodiment, the information regarding the increase or decrease in the level of at least one miRNA can be used to determine the treatment efficacy, as well as to tailor the treatment regimens of therapeutic interventions.

In one embodiment, the present methods are used for the titration of a subject's immunosuppression. Additionally, the inventive method can be utilized to determine whether the response to drug therapy indicates resolution of rejection risk. It can also be used to test whether the reduction of drug therapy increases the risk of rejection and whether drug therapy, if discontinued, should be resumed. This helps avoiding over-medication and/or under-medication of a given patient and duration of treatment can be tailored to the needs of the patient. The titration of immunosuppression can be after organ transplantation, or during a viral or bacterial infection. Further, the titration can be during a viral or bacterial infection after a subject has undergone organ transplantation. The method can include monitoring the response of a subject to one or more immunosuppressive agents, the withdrawal of an immunosuppressive agent, an antiviral agent, or an anti-bacterial agent.

Information gained by the methods described herein can be used to develop a personalized treatment plan for a transplant recipient. Accordingly, the invention further provides methods for developing personalized treatment plans for transplant recipients. The methods can be carried out by, for example, carrying out any of the methods of miRNA analysis described herein and, in consideration of the results obtained, designing a treatment plan for the patient whose transplant is assessed. If the levels of gene expression indicate that the patient is at risk for an undesirable clinical outcome (e.g., transplant rejection, developing delayed graft function, or compromised graft function), the patient is a candidate for treatment with an effective amount of an immunosuppressant. Depending on the level of miRNAs the patient may require a treatment regime that is more aggressive than a standard regime, or it may be determined that the patient is best suited for a standard regime. When so treated, one can treat or prevent transplant rejection (or, at least, prolong the time the transplanted organ functions adequately). Conversely, a different result (i.e., a different level of miRNAs) may indicate that the patient is not likely to experience an undesirable clinical outcome. In that event, the patient may avoid immunosuppressants. U.S. Pat. No. 8,741,557.

Pharmacologic agents for therapeutic interventions can include, but are not limited to, miRNA based therapeutics (including antisense oligonucleotides), an immunosuppressant or a combination thereof.

In various embodiments, the therapeutic intervention is a miRNA-based therapy. In some embodiments, the miRNA based therapeutic is an antisense oligonucleotide. The antisense oligonucleotides may be ribonucleotides or deoxyribonucleotides. In some embodiments, the miRNA based therapeutic is an antisense oligonucleotide targeting an miRNA selected from Table 1. The antisense oligonucleotide therapeutics may have at least one chemical modification (i.e., the oligonucleotide is chemically modified). For instance, suitable antisense oligonucleotides may be comprised of one or more conformationally constrained or bicyclic sugar nucleoside modifications, for example, locked nucleic acids (LNAs) in some embodiments, the miRNA based therapeutic is a chemically-modified antisense oligonucleotide. In some embodiments, the miRNA based therapeutic is a chemically-modified antisense oligonucleotide targeting a miRNA expressed in heart tissue.

Alternatively, the antisense oligonucleotides may comprise peptide nucleic acids (PNAs), which contain a peptide-based backbone rather than a sugar-phosphate backbone. Other chemical modifications that the antisense oligonucleotides may contain include, but are not limited to, sugar modifications, such as 2′-O-alkyl (e.g. 2′-O-methyl, 2′-O-methoxyethyl), 2′-fluoro, and 4′ thio modifications, and backbone modifications, such as one or more phosphorothioate, morpholino, or phosphonocarboxylate linkages (U.S. Pat. Nos. 6,693,187 and 7,067,641). For instance, antisense oligonucleotides, particularly those of shorter lengths (e.g., less than 15 nucleotides) can comprise one or more affinity enhancing modifications, such as, but not limited to, LNAs, bicyclic nucleosides, phosphonoformates, 2′ O alkyl and the like. Locked nucleic acids (LNAs) are modified nucleotides that contain an extra bridge between the 2′ and 4′ carbons of the ribose sugar moiety resulting in a locked conformation that confers enhanced thermal stability to oligonucleotides containing the LNAs. LNAs are described, for example, in U.S. Pat. No. 6,268,490, U.S. Pat. No. 6,316,198, U.S. Pat. No. 6,403,566, U.S. Pat. No. 6,770,748, U.S. Pat. No. 6,833,361, U.S. Pat. No. 6,998,484, U.S. Pat. No. 6,670,461, and U.S. Pat. No. 7,034,133.

In other embodiments, suitable antisense oligonucleotides are 2′-O-methoxyethyl S gapmers which contain 2′-O-methoxyethyl-modified ribonucleotides on both 5′ and 3′ ends with at least ten deoxyribonucleotides in the center. These gapmers are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the invention. Preferable antisense oligonucleotides useful for inhibiting the activity of miRNAs are about 5 to about 50 nucleotides in length, about 10 to about 30 nucleotides in length, about 8 to about 18 nucleotides, about 12 to 16 nucleotides, about 8 nucleotides or greater, or about 20 to about 25 nucleotides in length.

In certain embodiments, antisense oligonucleotides may comprise a sequence that is at least partially complementary to a mature miRNA sequence, e.g., at least about 75%, 80%, 85%, 90%, 95%, 96%, 97%, 98%, or 99% complementary to a mature miRNA sequence. In some embodiments, the antisense oligonucleotide may be substantially complementary to a mature miRNA sequence, that is at least about 95%, 96%, 97%, 98%, or 99% complementary to a target miRNA sequence. In one embodiment, the antisense oligonucleotide comprises a sequence that is 100% complementary to a mature miRNA sequence.

In other embodiments, the antisense oligonucleotides are antagomirs. Antagomirs are single-stranded, chemically-modified ribonucleotides that are at least partially complementary to the miRNA sequence. Antagomirs may comprise one or more modified nucleotides, such as 2′-O-methyl-sugar modifications. In some embodiments, antagomirs comprise only modified nucleotides. Antagomirs may also comprise one or more phosphorothioate linkages resulting in a partial or full phosphorothioate backbone. To facilitate in vivo delivery and stability, the antagomir may be linked to a steroid such as cholesterol, a fatty acid, a vitamin, a carbohydrate, a peptide or another small molecule ligand at its 3′ end. Antagomirs suitable for inhibiting miRNAs may be about 15 to about 50 nucleotides in length, about 18 to about 30 nucleotides in length, or about 20 to about 25 nucleotides in length. “Partially complementary” refers to a sequence that is at least about 75%, 80%, 85%, 90%, 95%, 96%, 97%, 98%, or 99% complementary to a target polynucleotide sequence. The antagomirs may be at least about 75%, 80%, 85%, 90%, 95%, 96%, 97%, 98%, or 99% complementary to a mature miRNA sequence. In some embodiments, the antagomir may be substantially complementary to a mature miRNA sequence, that is at least about 95%, 96%, 97%, 98%, or 99% complementary to a target polynucleotide sequence. In other embodiments, the antagomirs are 100% complementary to the mature miRNA sequence.

### Samples

Sampling methods are well known by those skilled in the art and any applicable techniques for obtaining biological samples of any type are contemplated and can be employed with the methods of the present invention. (See, e.g., Clinical Proteomics: Methods and Protocols, Vol. 428 in Methods in Molecular Biology, Ed. Antonia Vlahou (2008).)

The samples may be drawn before, during or after transplantation. The samples may be drawn at different time points during transplantation, and/or be drawn at different time points after transplantation.

When the sample is drawn after transplantation, it can be obtained from the subject at any point following transplantation. In some embodiments, the sample is obtained about 1 week, about 2 weeks, about 3 weeks, about 1 month, about 2 months, about 3 months, about 4 months, about 5 months, about 6 months, at least 1, 2, 3, or 6 months following transplantation. In some embodiments, the sample is obtained least 1, 2, 3, 4, 6 or 8 weeks following transplantation. In some embodiments, the sample is obtained at least 1, 2, 3, 4, 5, 6, or 7 days following transplantation. In some embodiments, the sample is obtained at least 10 minutes, 30 minutes, 1 hour, 6 hours, 12 hours, 18 hours or 24 hours after transplantation. In other embodiments, the sample is obtained at least one week following transplantation. In some embodiments, one or more miRNAs selected from Table 1 is measured between 1 and 8 weeks, between 2 and 7 weeks, at 1, 2, 3, 4, 5, 6, 7 or 8 weeks following transplantation.

### Kits

Another aspect of the invention is a kit containing a reagent or reagents for measuring at least one miRNA in a biological sample, instructions for measuring the at least one miRNA, and/or instructions for evaluating or monitoring transplant rejection in a patient based on the level of the at least one miRNA, and/or instructions for assessing an immunosuppressant therapy in a patient. In some embodiments, the kit contains reagents for measuring from 2 to about 20 human miRNAs, including at least 3, 4, 5, 6, 7, 8, 9, 10, 11, 12, 13, 14, 15 or more from Table 1.

In one embodiment, the kit reagent comprises a miRNA-specific primer and/or probe for reverse transcribing, amplifying, and/or hybridizing to one or more miRNAs described herein. Such kits can further comprise one or more normalization controls and/or a TaqMan probe specific for each miRNA of the kit.

Any of the compositions described herein may be comprised in a kit. In one embodiment, the kit contains a reagent for measuring at least one miRNA selected from Table 1 in a biological sample, instructions for measuring the at least one miRNA and instructions for evaluating or monitoring transplant rejection in a patient based on the level of the at least one miRNA. In some embodiments, the kit contains reagents for measuring the level of at least 2, 3, 4, 5, 6 or 10 miRNAs (or more), from Table 1. The kit may also be customized for determining the efficacy of therapy for transplant rejection, and thus provides the reagents for determining 50 or fewer, 40 or fewer, 30 or fewer, or 25 or fewer miRNAs, including the miRNAs of Table 1.

In certain embodiments, the kit can further contain one or more normalization controls. The one or more normalization controls are provided as one or more separate reagents for spiking samples or reactions. The normalization control can be added in a range of from about 0.1 fmol to about 5 mol. In some embodiments, the normalization control is added at about 0.1 fmol, 0.5 fmol, 1 fmol, 2 fmol, 3 fmol, 4 fmol or 5 fmol. In some embodiments, the at least one normalization control is a non-endogenous RNA or miRNA, or a miRNA not expressed in the sample.

The kit can further contain a TaqMan probe specific for each miRNA of the kit. In some embodiments, the TaqMan probe is specific for a miRNA selected from Table 1.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed (e.g., sterile, pharmaceutically acceptable buffer and/or other diluents). However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution may be an aqueous solution. The components of the kit may also be provided as dried powder(s). When reagents and/or components are provided as a dry powder, the powder can be reconstituted by the addition of a suitable solvent. It is envisioned that the solvent may also be provided in another container means.

Such kits may also include components that preserve or maintain the reagents or that protect against their degradation. Such components may be DNAse-free, RNAse-free or protect against nucleases (e.g., RNAses and DNAses). Such kits generally will comprise, in suitable means, distinct containers for each individual reagent or solution.

A kit will also include instructions for employing the kit components as well the use of any other reagent not included in the kit. Instructions may include variations that can be implemented.

The invention may also encompass biochips. Biochips contain a microarray of probes which are capable of hybridizing to the miRNAs described herein. The probes may either be synthesized first, with subsequent attachment to the biochip, or may be directly synthesized on the biochip.

The following are examples of the present invention and are not to be construed as limiting.

### Example 1

We studied serum levels of miRs associated with immune cell function (miR-155, 125b, 142-3p, 144, 223-3p, 27a and 101), myocardial damage (miR-1) and immunosuppression (let-7a). We hypothesized that these miRs would be associated with acute cellular rejection (ACR) and antibody-mediated rejection (AMR).

### Methods

Subject recruitment: Patients who received HTx at Columbia University Medical Center and gave informed consent were enrolled. Clinical data were obtained from patient charts. Samples were obtained from control subjects who gave informed consent.

Sample processing: Blood was obtained from HTx recipients >2 months post-HTx. Blood samples were centrifuged in EDTA-containing tubes at 1500× relative centrifugal force for 15 minutes. The plasma fraction was aspirated for further analysis. 200 μL of each plasma sample was then processed with an Exiqon miRCURY™ RNA Isolation Kit.

Rejection was defined by histology of cardiac biopsy specimens and graded according to ISHLT (The International Society for Heart and Lung Transplantation) guidelines (Table 2 and Table 3). AMR was defined by C4D⁺ positivity. 7 ACR patients had ISHLT 1R/1B rejection and 5 had 2R/3A rejection. 6 ACR and 5 AMR patients had donor-specific antibodies, respectively. Plasma was isolated by centrifugation and frozen at −80° C.

RNA isolation: RNA was isolated with Exiqon miRCURY Biolfluid RNA Isolation Kit according to manufacturer's instructions.

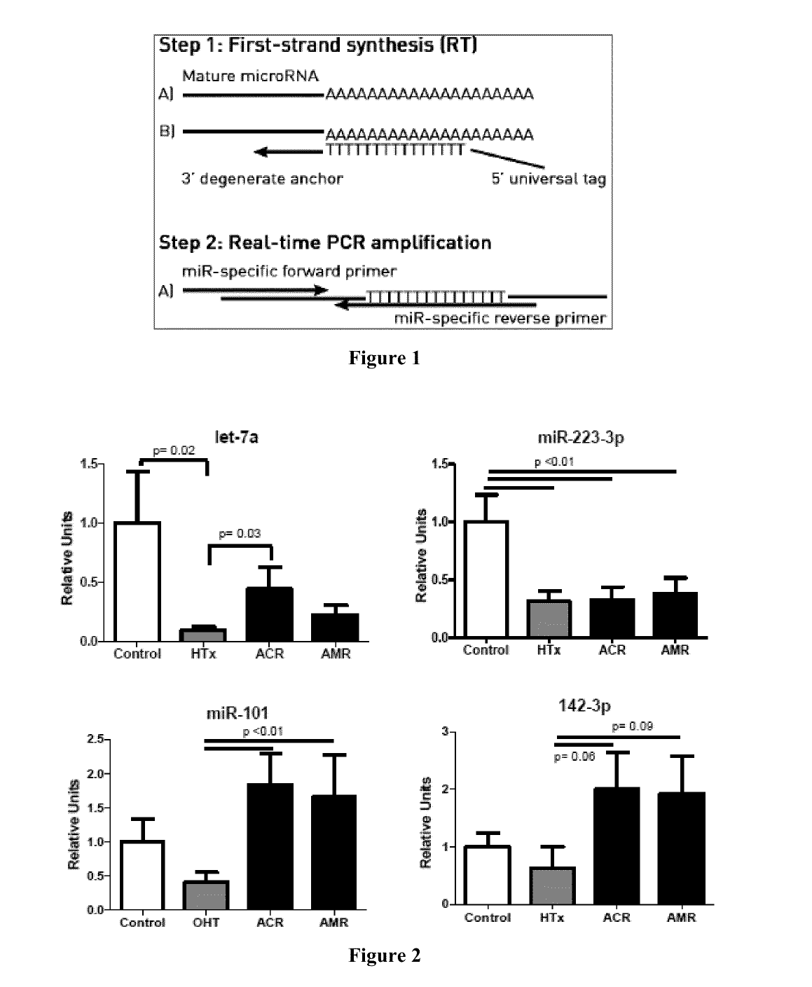

RT-PCR: RT and qPCR reactions were performed on a PikoReal Real-Time PCR System (Thermo Scientific) with LNA® primers and reagents (Exiqon) according to published protocols. The scheme of RT-PCR is shown in FIG. 1.

cDNA Synthesis: 2 μL of each eluate was mixed with 2 μL 5× reaction buffer, 5 μL nuclease-free water, and 1 μL enzyme mix, all components of the Exiqon Universal cDNA synthesis kit II (product #203301) at 4° C. The total reaction volume was 10 μL.

Each cDNA synthesis mixture was incubated for 60 minutes at 42° C. then heat-inactivated at 95° C. for 5 minutes and cooled to 4° C.

Quantitative PCR: Each cDNA product mixture was diluted 40× with nuclease-free water. 4 μL of each diluted cDNA product mixture was mixed with 5 μL of ExiLENT SYBR® Green master mix (product #203421) and 1 μL of an equal mixture of miRNA-specific forward and reverse primers at 4° C. The total reaction volume was 10 μL. Quantitative PCR was performed with a Thermo Scientific PikoReal™ Real-Time PCR System. The protocol consisted of 40 amplification cycles of 95° C. for 10 seconds then 60° C. for 1 minute with a ramp-rate of 1.6° C. and an optical read. For each sample miRNA levels were normalized to 5S RNA with the 2^{-ΔCq} method.

We analyzed samples from 12 healthy volunteers (“control”), 12 heart transplant recipients without evidence of rejection (“HTx”), 12 heart transplant recipients with acute cellular rejection (“ACR”), and 11 heart transplant recipients with antibody-mediated rejection (“AMR”). Baseline demographics of the subjects are shown in Table 4.

TABLE 4
Baseline Demographics

| | Control | OHT | ACR | AMR | p-value |
|---|---|---|---|---|---|
| N | 12 | 12 | 12 | 11 | |
| Age (years) | 44.3 ± 3.2 | 53.8 ± 3.2 | 47.5 ± 3.2 | 48.2 ± 3.3 | 0.2097 |
| Sex (M/F) | 6/6 | 9/3 | 5/7 | 7/4 | 0.3537 |
| Etiology of | NA | 6/2/4 | 6/1/4 | 5/3/4 | 0.8984 |
| Heart Failure | | | | | |
| (D/I/O) | | | | | |

Comorbidities

| | | | | | |
|---|---|---|---|---|---|
| DM | NA | 8 (67%) | 8 (73%) | 7 (58%) | 0.7649 |
| HTN | NA | 8 (67%) | 9 (82%) | 9 (75%) | 0.7047 |
| CAD | NA | 1 (8%) | 1 (9%) | 2 (17%) | 0.7877 |
| CKD | NA | 7 (58%) | 6 (55%) | 7 (58%) | 0.9782 |

Immunosuppressive Medications

| | | | | | |
|---|---|---|---|---|---|
| Tacrolimus | NA | 11 (92%) | 9 (75%) | 9 (82%) | 0.5329 |
| Steroids | NA | 12 (100%) | 12 (100%) | 11 (100%) | 1.000 |
| Cellcept | NA | 12 (100%) | 7 (67%) | 8 (64%) | 0.0659 |
| Cyclosporine | NA | 2 (40%) | 2 (40%) | 1 (20%) | 0.8380 |

D = dilated, I = ischemic, O = other. DM = diabetes mellitus, HTN = hypertension, CAD = coronary artery disease, CKD = chronic kidney disease. Continuous data displayed as mean ± standard deviation.

FIG. 2 shows miRNAs let-7a, miR-223-3p, miR-101 and miR-142-3p levels associated with HTx (without rejection) and transplant rejection (ACR and AMR).

Table 5 shows the relative units from RT-PCR for let-7a-3p, corresponding to its levels in various samples. Table 5 also contains the normalized mean for control, HTx, ACR and AMR samples, as well as standard deviation (SD) and standard error of the mean (SEM).

TABLE 5

| | let-7a-3p Relative Units | | | |
|---|---|---|---|---|
| | Control | HTx | ACR | AMR |
| Sample | 9.553629 | 1.400557 | 0.880248 | 1.390953 |
| Sample | 3.151561 | 2.259586 | 0.458886 | 0.956636 |
| Sample | 62.94634 | 0.488369 | 16.17905 | 0.001563 |
| Sample | 16.8661 | 0.154563 | 22.88053 | 2.86008 |
| Sample | 1.835321 | 0.053159 | 0.296506 | 4.044763 |
| Sample | 6.993628 | 1.797573 | 4.456968 | 5.840326 |
| Sample | 4.646151 | 3.044126 | 4.743869 | |
| Sample | 1.450031 | 0.495181 | 2.008422 | |
| Sample | 4.101161 | 0.671745 | 3.521216 | |
| Sample | 2.668552 | 0.161152 | 0.360051 | |
| Sample | 14.99124 | 5.227213 | 2.197828 | |
| Sample | 4.810094 | 0.256302 | 1.129737 | |
| Sample | | 0.053494 | | |
| Sample | | 0.667166 | | |
| Sample | | 0.106988 | | |
| Sample | | 0.071586 | | |
| Sample | | 0.091241 | | |
| Mean | 11.16782 | 1.000000 | 4.92611 | 2.51572 |
| (normalized) | | | | |
| Change | | | Increase about | Increase about |
| Compared | | | 3.9 fold | 1.5 fold |
| to HTx | | | | |
| SD | 17.05353 | 1.401232 | 7.136291 | 2.169165 |
| SEM | 4.922931 | 0.339849 | 2.06007 | 0.885558 |

Table 6 shows the relative units from RT-PCR for miR-223-3p, corresponding to its levels in various samples. Table 6 also contains the normalized mean for control, HTx, ACR and AMR samples, as well as standard deviation (SD) and standard error of the mean (SEM).

TABLE 6

| | miR-223-3p Relative Units | | | |
|---|---|---|---|---|
| | Control | HTx | ACR | AMR |
| Sample | 4.837749 | 2.646955 | 0.776116 | 1.090000 |
| Sample | 1.398937 | 3.061708 | 0.158693 | 0.186127 |
| Sample | 8.249641 | 0.128914 | 2.721354 | 0.000353 |
| Sample | 7.332629 | 0.210862 | 0.216773 | 1.045605 |
| Sample | 1.201085 | 0.103985 | 1.640734 | 2.369082 |
| Sample | 5.149127 | 3.327246 | 3.769401 | 2.610526 |
| Sample | 1.552233 | 3.126021 | 0.418801 | |
| Sample | 0.464674 | 0.744506 | 0.861149 | |
| Sample | 3.39718 | 0.670974 | 0.160909 | |
| Sample | 1.758469 | 0.033602 | 0.652632 | |
| Sample | 2.006017 | 1.287274 | 0.184842 | |
| Sample | 1.201085 | 0.545016 | | |
| Sample | | 0.109896 | | |
| Sample | | 0.588191 | | |
| Sample | | 0.212308 | | |
| Sample | | 0.027659 | | |
| Sample | | 0.174883 | | |
| Mean | 3.212402 | 1.000000 | 1.051037 | 1.216949 |
| (normalized) | | | | |
| Change | | | Increase | Increase |
| Compared | | | about 5% | about 22% |
| to HTx | | | | |
| SD | 2.594255 | 1.216786 | 0.347898 | 1.082372 |
| SEM | 0.748897 | 0.295114 | 0.104895 | 0.441876 |

Table 7 shows the relative units from RT-PCR for miR-101-3p, corresponding to its levels in various samples. Table 7 also contains the normalized mean for control, HTx, ACR and AMR samples, as well as standard deviation (SD) and standard error of the mean (SEM).

TABLE 7

| | miR-101-3p Relative Units | | | |
|---|---|---|---|---|
| | Control | OHT | ACR | AMR |
| Sample | 2.916229 | 0.296094 | 0.630286 | 4.210823 |
| Sample | 4.513043 | 1.296034 | 0.643539 | 0.455051 |
| Sample | 8.248339 | 0.109876 | 9.344414 | 7.857671 |
| Sample | 2.44E−05 | 1.617876 | 11.18973 | 3.640405 |
| Sample | 0.000195 | 1.438028 | 0.011007 | |
| Sample | 1.408448 | 0.803349 | 4.329217 | |
| Sample | 3.040065 | 0.319548 | 7.857671 | |
| Sample | 3.019076 | 4.77038 | 6.840501 | |
| Sample | 1.260596 | 0.330824 | 7.433812 | |
| Sample | 4.88E−05 | 0.000000 | 1.200899 | |
| Sample | | 0.849159 | 3.468001 | |
| Sample | | 0.670873 | 0.666236 | |
| Sample | | 0.497956 | | |
| Mean | 2.440607 | 1.000000 | 4.467943 | 4.040988 |
| (normalized) | | | | |
| Change | | | Increase | Increase |
| Compared | | | about 3.5 | about 3 |
| to HTx | | | fold | fold |
| SD | 2.566257 | 1.241553 | 3.934021 | 3.033992 |
| SEM | 0.811522 | 0.344345 | 1.135654 | 1.516996 |

Table 8 shows the relative units from RT-PCR for miR-142-3p, corresponding to its levels in various samples. Table 8 also contains the normalized mean for control, HTx, ACR and AMR samples, as well as standard deviation (SD) and standard error of the mean (SEM).

TABLE 8

| | miR-142-3p Relative Units | | | |
|---|---|---|---|---|
| | Control | HTx | ACR | AMR |
| Sample | 0.115947 | 0.10164 | 0.225566 | 0.536498 |
| Sample | 0.376741 | 0.435762 | 0.23678 | 1.080451 |
| Sample | 4.174579 | 0.003886 | 12.39451 | 6.371487 |
| Sample | 2.754185 | 0.041002 | 6.59618 | 2.268332 |
| Sample | 0.717795 | 0.514641 | 2.871147 | 2.044337 |
| Sample | 1.157998 | 0.299702 | 3.120188 | 6.027785 |
| Sample | 1.321006 | 0.490262 | 1.894255 | |
| Sample | 0.859533 | 0.206136 | 3.185743 | |
| Sample | 2.66038 | 0.651399 | 5.103997 | |
| Sample | 1.31187 | 0.451132 | 0.514641 | |
| Sample | 0.441853 | 0.061717 | 1.42566 | |
| Sample | 3.141886 | 0.24176 | 0.480174 | |
| Sample | | 0.020921 | | |
| Sample | | 0.077039 | | |
| Sample | | 3.367389 | | |
| Sample | | 9.79217 | | |
| Sample | | 0.243442 | | |
| Mean | 1.586148 | 1.000000 | 3.170737 | 3.054815 |
| (normalized) | | | | |
| Change | | | Increase | Increase |
| Compared | | | about 2.2 | about 2.1 |
| to HTx | | | fold | fold |
| SD | 1.28569 | 2.395583 | 3.527715 | 2.518671 |
| SEM | 0.371147 | 0.581014 | 1.018364 | 1.028243 |

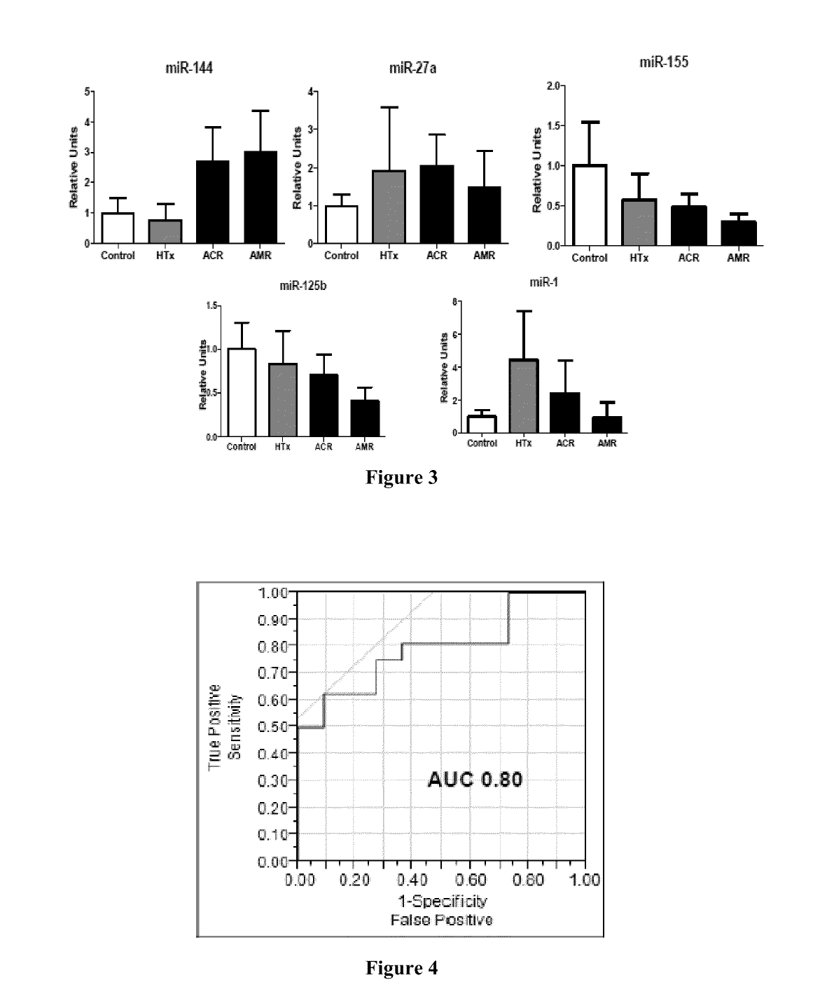

FIG. 3 shows miRNAs miR-144, miR-27a, miR-155, miR-125b, and miR-1 levels associated with HTx (without rejection) and transplant rejection (ACR and AMR).

Table 9 shows the relative units from RT-PCR for miR-144-3p, corresponding to its levels in various samples. Table 9 also contains the normalized mean for control, HTx, ACR and AMR samples, as well as standard deviation (SD) and standard error of the mean (SEM).

TABLE 9

| | miR-144-3p Relative Units | | | |
|---|---|---|---|---|
| | Control | HTx | ACR | AMR |
| Sample | 0.714876 | 0.024276 | 0.051324 | 3.825781 |
| Sample | 5.262515 | 0.223097 | 0.176254 | 3.77311 |
| Sample | 0.008524 | 0.173832 | 11.67826 | 0.223097 |
| Sample | 0.52331 | 0.005622 | 1.306545 | 10.23724 |
| Sample | 2.80064 | 0.209611 | 0.006114 | 1.521778 |
| Sample | 0.740085 | 0.404934 | 1.352623 | |
| Sample | 0.313335 | 0.804273 | 14.88465 | |
| Sample | 6.48E−05 | 1.480157 | 7.546219 | |
| Sample | | 0.195569 | 2.455049 | |
| Sample | | 1.3E−05 | 0.631024 | |
| Sample | | 0.120383 | 1.34327 | |
| Sample | | 0.000000 | 0.471635 | |
| Sample | | 9.820207 | | |
| Sample | | 0.538026 | | |
| Mean | 1.295419 | 1.000000 | 3.491914 | 3.916201 |
| (normalized) | | | | |
| Change | | | Increase | Increase |
| Compared | | | about 2.5 | about 2.9 |
| to HTx | | | fold | fold |
| SD | 1.833952 | 2.570617 | 5.050483 | 3.852158 |
| SEM | 0.6484 | 0.687026 | 1.457949 | 1.722737 |

Table 10 shows the relative units from RT-PCR for miR-27a-3p, corresponding to its levels in various samples. Table 10 also contains the normalized mean for control, HTx, ACR and AMR samples, as well as standard deviation (SD) and standard error of the mean (SEM).

TABLE 10

| | miR-27a-3p Relative Units | | | |
|---|---|---|---|---|
| | Control | HTx | ACR | AMR |
| Sample | 0.000254 | 0.063899 | 0.277768 | 0.297709 |
| Sample | 0.136973 | 0.567214 | 0.02108 | 0.064344 |
| Sample | 0.81336 | 0.000746 | 3.321798 | 0.183262 |
| Sample | 1.695799 | 0.032395 | 4.383138 | 2.773991 |
| Sample | 0.05962 | 0.000554 | 0.096185 | 0.490379 |
| Sample | 0.100967 | 0.366519 | 1.455953 | |
| Sample | 0.31035 | 0.871736 | 2.116917 | |
| Sample | 0.871736 | 0.132305 | 0.316872 | |
| Sample | 1.12659 | 0.046455 | 0.629362 | |
| Sample | 0.953938 | 4.14E−05 | 0.007771 | |
| Sample | 0.05563 | 0.227189 | 0.086088 | |
| Sample | 0.091631 | 0.055242 | 0.019132 | |
| Sample | | 0.006139 | | |
| Sample | | 3.11E−05 | | |
| Sample | | 0.015755 | | |
| Sample | | 0.0013 | | |
| Sample | | 15.58368 | | |
| Sample | | 0.028794 | | |
| Mean | 0.518071 | 1.000000 | 1.061005 | 0.761937 |
| (normalized) | | | | |
| Change | | | Increase | Decrease |
| Compared | | | about 6% | about 24% |
| to HTx | | | | |
| SD | 0.55563 | 3.647302 | 1.474935 | 1.135684 |
| SEM | 0.160396 | 0.859677 | 0.425777 | 0.507893 |

Table 11 shows the relative units from RT-PCR for miR-155-3p, corresponding to its levels in various samples. Table 11 also contains the normalized mean for control, HTx, ACR and AMR samples, as well as standard deviation (SD) and standard error of the mean (SEM).

TABLE 11

| | miR-155-3p Relative Units | | | |
|---|---|---|---|---|
| | Control | HTx | ACR | AMR |
| Sample | 1.659255 | 0.025221 | 0.000384 | 0.4933 |
| Sample | 1.12548 | 0.346408 | 1.729717 | 0.013056 |
| Sample | 10.27127 | 0.038748 | 1.919249 | 0.650908 |
| Sample | 1.485083 | 0.094775 | 0.835396 | 0.895368 |
| Sample | 0.341643 | 0.12504 | 0.939875 | |
| Sample | 0.10516 | 0.067477 | 0.211768 | |
| Sample | 1.117713 | 0.033965 | 0.244948 | |
| Sample | 0.650908 | 0.000384 | | |
| Sample | 0.126785 | 0.075907 | | |
| Sample | 0.570603 | 0.727252 | | |
| Sample | | 5.171365 | | |
| Sample | | 0.100866 | | |
| Sample | | 6.192593 | | |
| Mean | 1.74539 | 1.000000 | 0.840191 | 0.513158 |
| (normalized) | | | | |
| Change | | | Decrease | Decrease |
| Compared | | | about 16% | about 49% |
| to HTx | | | | |
| SD | 3.044173 | 2.09739 | 0.754993 | 0.372181 |
| SEM | 0.962652 | 0.581711 | 0.285361 | 0.186091 |

Table 12 shows the relative units from RT-PCR for miR-125b-3p, corresponding to its levels in various samples. Table 12 also contains the normalized mean for control, HTx, ACR and AMR samples, as well as standard deviation (SD) and standard error of the mean (SEM).

TABLE 12

| | miR-125b-3p Relative Units | | | |
|---|---|---|---|---|
| | Control | HTx | ACR | AMR |
| Sample | 2.308784 | 0.126497 | 0.585253 | 0.705697 |
| Sample | 1.298752 | 0.32695 | 0.118024 | 0.197122 |
| Sample | 2.803302 | 0.761617 | 1.263241 | 0.029506 |
| Sample | 0.827676 | 0.244365 | 1.335269 | 0.206913 |
| Sample | 0.66302 | 0.014345 | 0.449732 | 0.516605 |
| Sample | 4.190523 | 0.589327 | 0.194401 | 1.298752 |
| Sample | 0.434415 | 0.425475 | 1.632557 | |
| Sample | 0.13938 | 0.386118 | 3.427432 | |
| Sample | 1.154386 | 0.931174 | 0.509486 | |
| Sample | 0.278772 | 0.244365 | 0.26191 | |
| Sample | 0.081171 | 0.172793 | 0.296713 | |
| Sample | 0.201256 | 0.141334 | 0.08521 | |
| Sample | | 0.046296 | | |
| Sample | | 0.649382 | | |
| Sample | | 0.26191 | | |
| Sample | | 0.038662 | | |
| Sample | | 7.245728 | | |
| Sample | | 0.589327 | | |
| Sample | | 5.804336 | | |
| Mean | 1.198453 | 1.000000 | 0.846602 | 0.492432 |
| (normalized) | | | | |
| Change | | | Decrease | Decrease |
| Compared | | | about 15% | about 51% |
| to HTx | | | | |
| SD | 1.278883 | 1.978342 | 0.963441 | 0.464596 |
| SEM | 0.369182 | 0.453863 | 0.278121 | 0.189671 |

Table 13 shows the relative units from RT-PCR for miR-125b-3p, corresponding to its levels in various samples. Table 13 also contains the normalized mean for control, HTx, ACR and AMR samples, as well as standard deviation (SD) and standard error of the mean (SEM).

TABLE 13

| | miR-1 Relative Units | | | |
|---|---|---|---|---|
| | Control | HTx | ACR | AMR |
| Sample | 0.001478 | 0.03145 | 6.76E−06 | 0.009416 |
| Sample | 0.000604 | 0.243036 | 2.78802 | 9.47E−05 |
| Sample | 0.005445 | 0.000104 | 2.93E−05 | 0.000194 |
| Sample | 0.800649 | 1.108987 | 0.222095 | 0.846301 |
| Sample | 0.444187 | 0.090825 | 0.248143 | |
| Sample | 0.238035 | 0.037661 | 0.016281 | |
| Sample | 0.090825 | 0.000151 | | |
| Sample | 0.029755 | 0.000996 | | |
| Sample | 0.417324 | 6.817493 | | |
| Sample | | 1.669297 | | |
| Mean | 0.225367 | 1.000000 | 0.545762 | 0.214001 |
| (normalized) | | | | |
| Change | | | Decrease | Decrease |
| Compared | | | about 46% | about 79% |
| to HTx | | | | |
| SD | 0.278661 | 2.123005 | 1.104272 | 0.421556 |
| SEM | 0.092887 | 0.671353 | 0.450817 | 0.210778 |

FIG. 4 shows a receiver operating characteristic (ROC) curve which suggests that a combination of let-7a, miR-101 and miR-144 can distinguish between HTx (without rejection) and transplant rejection.

### Conclusion

MiRNAs hold promise as biomarkers of HTx rejection. We assessed whether circulating miRNAs can serve as biomarkers of organ rejection among HTx patients. Study of serum miRNAs related to immune function may aid in diagnosis of cardiac allograft rejection. Our data show that miR-101 and 223-3p and let-7a have diagnostic potential for identifying patients with ACR and AMR.

### Example 2

We studied serum levels of miRs associated with immune cell function (miR-155, 125b, 142-3p, 144, 223-3p, 27a and 101), myocardial damage (miR-1) and immunosuppression (let-7a). Blood was obtained from HTx recipients >2 months post-HTx. Rejection was defined by histology of cardiac biopsy specimens and graded according to ISHLT guidelines. MiRs were analyzed with RT-PCR and normalized to 5S rRNA.

### Sample Processing

Blood samples were centrifuged in EDTA-containing tubes at 1500× relative centrifugal force for 15 minutes. The plasma fraction was aspirated for further analysis.

200 μL of each plasma sample was then processed with an Exiqon miRCURY™ RNA Isolation Kit.

cDNA Synthesis

2 μL of each eluate was mixed with 2 μL 5× reaction buffer, 5 μL nuclease-free water, and 1 μL enzyme mix, all components of the Exiqon Universal cDNA synthesis kit II (product #203301) at 4° C. The total reaction volume was 10 μL.

Each cDNA synthesis mixture was incubated for 60 minutes at 42° C. then heat-inactivated at 95° C. for 5 minutes and cooled to 4° C.

### Quantitative PCR

Each cDNA product mixture was diluted 40× with nuclease-free water. 4 μL of each diluted cDNA product mixture was mixed with 5 μL of ExiLENT SYBR® Green master mix (product #203421) and 1 μL of an equal mixture of miRNA-specific forward and reverse primers at 4° C. The total reaction volume was 10 μL. Quantitative PCR was performed with a Thermo Scientific PikoReal™ Real-Time PCR System. The protocol consisted of 40 amplification cycles of 95° C. for 10 seconds then 60° C. for 1 minute with a ramp-rate of 1.6° C. and an optical read. For each sample miRNA levels were normalized to 5S with the 2^{-Δcq} method.

### Results

We analyzed samples from 12 healthy volunteers (44±6 yrs), 12 HTx recipients without evidence of rejection (54±11 yrs), 11 patients with acute cellular rejection (ACR; 48±12 yrs), and 6 patients with antibody-mediated rejection (AMR; defined by C4D⁺ immunohistochemistry; 48±13 yrs).

The ACR group included 7 patients with ISHLT 1R/1B rejection and 4 with 2R/3A. 6 of 11 ACR and 5 of 6 AMR patients had evidence of donor-specific antibodies. Let-7a levels were significantly lower among the HTx group vs. controls (0.09±0.13 vs. 1.00±1.53 RU; p=0.02) and increased only among the ACR group (0.44±0.64, p=0.03) vs. the HTx group. MiR-223-3p was lower among HTx (0.31±0.38), ACR (0.33±0.37) and AMR (0.38±0.37) vs. control (1.00±0.87, p<0.01). MiR-101 levels were increased among ACR (1.83±1.61, p<0.01) and AMR groups (1.66±1.24, p<0.01) vs. HTx (0.41±0.51). There were trends towards higher miR-142-3p among ACR (p=0.06) and AMR (p=0.09) vs. the HTx group. Combination of let-7a and miR-101 and miR-144 identified patients with acute rejection compared to stable HTx patients (ROC AUC 0.82).

### Conclusion

Circulating miRNAs related to immune function may aid in diagnosis of cardiac allograft rejection. Our data show that miR-101 and 223-3p and let-7a have diagnostic potential for identifying patients with ACR and AMR.

### Example 3

We assessed serum levels of the cardiac miRNAs miR-1 and miR-101 and the immunologic miRNAs miR-155, miR-125b, miR-142-3p, miR-144, miR-223-3p, miR-27a, and let-7a by RT-PCR. Subjects included 12 healthy volunteers, 13 HTx recipients without rejection (mean age 52.3 yrs; 10 men, 3 women), 11 patients with ACR (mean age 48.5; 6M/5W) and 5 patients with AMR (mean age 45.2; 2M/3W). Blood samples were obtained >2 months post-HTx. All HTx recipients were receiving tacrolimus, prednisone and mycophenolate. Levels of miRs were normalized to 5S rRNA.

Results: Levels of miR-101 were significantly higher in ACR (p<0.05) compared to no rejection. Levels of miR-144 were significantly increased in both ACR and AMR. Levels of miR-223-3p were suppressed in all HTx recipients compared to control, regardless of rejection status. Let-7a, which is known to be suppressed by tacrolimus, was significantly suppressed among all HTx recipients and there were trends towards an increase among patients with ACR and AMR.

Conclusion: Dynamics in circulating miRs are detectable following HTx. Further studies will analyze the role of miRs as ancillary markers of immunosuppression.

### Example 4

We isolated microRNA from serum obtained from 10 patients at least 3 months after uncomplicated HTx (mean age 55±13 years) and 10 healthy controls (44±16 years). All HTx recipients were receiving tacrolimus, prednisone and mycophenolate. We performed 2 parallel isolations of each sample. We prepared a cDNA library of each preparation and conducted RNA deep sequencing.

Results: In comparison to control subjects, there were significant decreases among post-HTx patients of let-7 family members, which are known to be suppressed by tacrolimus treatment. Across both preparations, there were consistent changes in levels of miR-15b*, -23a, -99b, -126, -191, -199a-5p, -425*, and -766 among HTx patients compared to controls (all p<0.05). Gene ontology analysis reveals roles for targets of altered miRs in B- and T-cell function and other immunologic processes.

Conclusion: Dynamics in circulating miRs are detectable following HTx among stable patients.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

1. A method for diagnosing or predicting transplant rejection in a subject who has received a transplant, the method comprising the steps of:
(a) obtaining a sample from the subject;
(b) assaying the level of one or more miRNAs in the sample, wherein the one or more miRNAs are selected from Table 1 (SEQ ID NOs: 1-508);
(c) comparing the level obtained in step (b) with the level of the one or more miRNAs in a control sample, and
(d) diagnosing or predicting transplant rejection if the level of at least one miRNA obtained in step (b) increases or decreases by at least 5% compared to its level in the control sample.

2. The method of claim 1, wherein the transplant is a heart transplant, a kidney transplant, a pancreas transplant, a liver transplant, a lung transplant, an intestine transplant, or a combination thereof.

3. The method of claim 1, wherein the one or more miRNAs are selected from the group consisting of miR-1, miR-101, miR-155, miR-125b, miR-142-3p, miR-144, miR-223-3p, miR-27a, let-7a, a let-7 family member, miR-15b*, miR-23a, miR-99b, miR-126, miR-191, miR-199a-5p, miR-425*, and miR-766, or combinations thereof.

4. The method of claim 3, wherein the one or more miRNAs are selected from the group consisting of let-7a, miR-101, miR-144, or combinations thereof.

5. The method of claim 1, wherein step (d) is diagnosing transplant rejection if the level of at least one miRNA obtained in step (b) increases or decreases by at least 15% compared to its level in the control sample.

6. The method of claim 1, wherein step (d) is diagnosing transplant rejection if the level of at least one miRNA obtained in step (b) increases by at least 1.5 fold compared to its level in the control sample.

7. The method of claim 1, wherein the sample is a plasma, serum or blood sample.

8. The method of claim 1, wherein the subject is treated with an immunosuppressant.

9. The method of claim 1, wherein the subject's existing immunosuppressive regimen is modified or maintained.

10. The method of claim 1, wherein the level of the one or more microRNAs is determined by RNA sequencing, microarray profiling or real-time PCR.

11. The method of claim 1, wherein the control sample is from a subject who has received a transplant without rejection or from a plurality of subjects who have received a transplant without rejection.

12. The method of claim 1, wherein the subject is human.

13. A method for treating a subject with transplant rejection or an increased risk of transplant rejection, the method comprising the steps of:
(a) obtaining a sample from the subject after a transplant;
(b) assaying the level of one or more miRNAs in the sample, wherein the one or more miRNAs are selected from Table 1 (SEQ ID NOs: 1-508);
(c) comparing the level obtained in step (b) with the level of the one or more miRNAs in a control sample, and
(d) treating the subject for transplant rejection or an increased risk of transplant rejection, if the level of at least one miRNA obtained in step (b) increases or decreases by at least 5% compared to its level in the control sample.

14. The method of claim 13, wherein the transplant is a heart transplant, a kidney transplant, a pancreas transplant, a liver transplant, a lung transplant, an intestine transplant, or a combination thereof.

15. The method of claim 13, wherein the one or more miRNAs are selected from the group consisting of miR-1, miR-101, miR-155, miR-125b, miR-142-3p, miR-144, miR-223-3p, miR-27a, let-7a, a let-7 family member, miR-15b*, miR-23a, miR-99b, miR-126, miR-191, miR-199a-5p, miR-425*, and miR-766, or combinations thereof.

16. The method of claim 15, wherein the one or more miRNAs are selected from the group consisting of let-7a, miR-101, miR-144, or combinations thereof.

17. The method of claim 13, wherein step (d) is treating the subject for transplant rejection or an increased risk of transplant rejection, if the level of at least one miRNA obtained in step (b) increases or decreases by at least 15% compared to its level in the control sample.

18. The method of claim 13, wherein the sample is a plasma, serum or blood sample.

19. The method of claim 13, wherein the subject is treated with an immunosuppressant.

20. The method of claim 13, wherein the level of the one or more microRNAs is determined by RNA sequencing, microarray profiling or real-time PCR.

21. The method of claim 13, wherein the control sample is from a subject who has received a transplant without rejection or from a plurality of subjects who have received a transplant without rejection.

22. The method of claim 13, wherein the subject is human.

23. A kit comprising:
miRNA-specific primers for reverse transcribing or amplifying one or more miRNAs selected from Table 1 (SEQ ID NOs: 1-508), in a plasma or serum sample from a subject who has received a transplant; and
instructions for measuring the one or more miRNAs for diagnosing or predicting transplant rejection in the subject.

24. The kit of claim 23, wherein the kit comprises miRNA-specific primers for one or more miRNAs selected from miR-1, miR-101, miR-155, miR-125b, miR-142-3p, miR-144, miR-223-3p, miR-27a, let-7a, a let-7 family member, miR-15b*, miR-23a, miR-99b, miR-126, miR-191, miR-199a-5p, miR-425*, and miR-766.
